(19) United States
(12) Patent Application Publication
(10) Pub. No.: US 2016/0345827 A1
(43) Pub. Date: Dec. 1, 2016

(54) TWO-PHOTON MICROSCOPY IMAGING RETINA CELL DAMAGE

(71) Applicant: POLGENIX, INC., Cleveland, OH (US)

(72) Inventors: Krzysztof Palczewski, Cleveland, OH (US); Grazyna Palczewski, Cleveland, OH (US)

(21) Appl. No.: 15/117,089

(22) PCT Filed: Feb. 5, 2015
(86) PCT No.: PCT/US2015/014634

§ 371 Date: Aug. 5, 2016

Related U.S. Application Data
(60) Provisional application No. 61/935,975, filed on Feb. 5, 2014.

(51) Int. Cl. A61B 3/12; A61B 5/00; A61B 3/14

(52) U.S. Cl. CPC A61B 3/1225; A61B 3/14; A61B 5/0071; A61B 5/4848; A61B 2503/40

(57) ABSTRACT

A method of determining retinal degeneration of photoreceptors and/or the retinal pigment epithelium (RPE) of a subject includes measuring two-photon induced fluorescence inner and/or outer segments of the photoreceptor cells and/or retinal pigment epithelium to assess photoreceptor cell death and retinal pigment epithelium cell death or degeneration.

### RELATED APPLICATION

This application claims priority from U.S. Provisional Application No. 61/935,975, filed Feb. 5, 2014, the subject matter of which is incorporated herein by reference in its entirety.

### GOVERNMENT FUNDING

This invention was made with government support under Grant No. R01EY008061, R24EY021126, R01EY009339, R01EY022606, R01EY022658, K08EY019031, K08EY019880, P30EY011373, awarded by The National Institutes of Health and R44AG043645 awarded by National Institute on Aging, and 5T32EY007157 and 5T32DK007319, awarded by The National Institutes of Health institutional training grants. The United States government has certain rights to the invention.

### BACKGROUND

In recent years, dramatic progress has been made in discovering genetic and environmental factors contributing to retinal diseases. Imaging modalities such as scanning laser ophthalmoscopy (SLO) and optical coherence tomography along with classic histological methods and functional techniques, such as electroretinography (ERG) and electrophysiological recordings, have facilitated characterization of retinal defects. Concurrently, molecular understanding of the chemistry and biology of vision has paved the way for the first successful treatment of inherited retinal diseases, such as Leber congenital amaurosis or the advanced exudative form of age-related macular degeneration (AMD). However, identifying the cell type where the pathology originates and understanding the underlying pathological mechanisms have remained a challenge, impeding progress toward development of therapies effective against several common retinal diseases.

### SUMMARY

Embodiments described herein relate to a method of determining and/or measuring retinal degeneration of photoreceptors of a retina of a subject. The method includes irradiating the retina of the subject with short pulse light from a laser having a wavelength in the range of 600 nm to 1000 nm to stimulate two-photon induced fluorescence. Two-photon induced fluorescence is then detected from inner and/or outer segments of the photoreceptor cells using a photon detector. An image of the detected fluorescence of the inner and/or outer segments of the photoreceptors is generated. The image is then compared to a reference image to assess photoreceptor cell death.

In some embodiments, an increase in the amount or spatial localization of the fluorescence of the generated image compared to the reference image can be indicative of an increased risk of photoreceptor cell death.

In some embodiments, a three dimensional image of the photoreceptor outer segment can be generated based on the detected fluorescence to determine the shape and/or volume of the outer segment of the photoreceptor. An increase in volume of the photoreceptor outer segment compared to a reference volume of a photoreceptor can be indicative of an increased risk of photoreceptor death. The increased volume of the photoreceptor outer segment compared to the reference volume can be at least about 50%, at least about 75%, at least about 100%, at least about 150%, at least about 200%, at least about 300% or more.

In some embodiments, the light used to irradiate the retina has a wavelength in the range of about 710 nm to about 750 nm (e.g., about 730 nm).

In other embodiments, the method can further include administering a therapeutic agent to the subject prior to irradiating the retina of the subject with short pulse light from the laser, and comparing the image to a reference image to assess the effect of the agent on inhibiting photoreceptor cell death. The therapeutic agent can include, for example, at least one of a Gs or Gq coupled serotonin receptor antagonist, an alpha 1 adrenergic antagonist, an alpha-2 adrenergic receptor agonist, and adenylyl cyclase inhibitor, an M3 receptor antagonist, a PLC inhibitor, or a primary amine, which forms transient shiff-bases with all-trans retinal in the eye.

The subject can be, for example, human or a genetically engineered animal. In one example the genetically engineered animal is a genetically engineered Abca^{-/-}Rdhe^{-/-} mouse.

In some embodiments, the retina of the subject can be irradiated with light effective to induce retinal degeneration prior to irradiating the retina to stimulate two photon induced fluorescence. For example, the retina of the subject can be photo-bleached prior to irradiating the retina to stimulate two photon induced fluorescence.

In other embodiments, the laser can be directed to a deformable minor prior to irradiating a focal area or volume of the retina. The deformable minor can provide fine focus adjustment and aberration correction of the laser on focal volume of the retina. The shape of the deformable minor can be controlled by an image quality metric feedback without the use of a wavefront sensor. A plurality of Zernike nodes can be used as basis functions for deformation of the deformable minor as well as focus and excitation of the laser. In some embodiments, the Zernike nodes can be sequentially optimized or optimized using a stochastic parallel gradient descent method.

In other embodiments, the retina of the subject can be irradiated with light from the laser having a pulse length in the range of 10 fs to 100 fs and a repetition frequency in the range of 76 MHz to 100 MHz.

Still other embodiments relate to a method of determining retinal degeneration of the retinal pigment epithelium of a subject. The method includes irradiating the retina of the subject with short pulse light from a laser having a wavelength in the range of 600 nm to 1000 nm to stimulate two-photon induced fluorescence of retinoid cycle fluorophores of the retinal pigment epithelium (RPE). The retinoid cycle fluorophores can include all-trans-retinal condensation products. Two-photon induced fluorescence of retinoid cycle fluorophores of the retinal pigment epithelium (RPE) is detected using a photon detector. An image of the detected fluorescence of the retinoid cycle fluorophores of retinal pigment epithelium (RPE) is generated. The image is then compared to a reference image to assess retinal degeneration.

In some embodiments, an increase in the amount or spatial localization of the fluorescence of the generated image compared to the reference image can be indicative of an increased risk of retinal degeneration.

In some embodiments, a three dimensional image of the retinoid cycle fluorophores in the retinal pigment epithelium is generated based on the detected fluorescence to determine the amount or spatial localization of the retinoid cycle fluorophores in the retinal pigment epithelium. The light used to irradiate the retina has a wavelength in the range of about 840 nm to about 870 nm (e.g., about 850 nm).

In other embodiments, the method can further include administering a therapeutic agent to the subject prior to irradiating the retina of the subject with short pulse light from the laser, and comparing the image to a reference image to assess the effect of the compound on inhibiting photoreceptor cell death. The therapeutic agent can include at least one of a Gs or Gq coupled serotonin receptor antagonist, an alpha 1 adrenergic antagonist, an alpha-2 adrenergic receptor agonist, and adenylyl cyclase inhibitor, an M3 receptor antagonist, a PLC inhibitor, or a primary amine, which forms transient shiff-bases with all-trans retinal in the eye.

Other embodiments described herein relate to a method of determining retinal degeneration in an subject that includes measuring two-photon induced fluorescence of a retina irradiated with short pulse light from a laser having a wavelength in the range of about 710 nm to about 750 nm and a laser having a wavelength in the range of about 830 nm to about 870 nm. The measured fluorescence of the retina irradiated with light having a wavelength in the range of about 710 nm to about 750 nm is compared with the measured fluorescence of the retina irradiated with light having a wavelength in the range of about 830 nm to about 870 nm to assess pathological changes in the retina.

In some embodiments, an increase in the ratio of measured fluorescence induced with light having a wavelength in the range of about 710 nm to about 750 nm to measured fluorescence induced with light having a wavelength in the range of about 830 nm to about 870 nm in photoreceptor cells compared to a reference ratio is indicative of increased risk of photoreceptor cell death.

In other embodiments, decrease in the ratio of measured fluorescence induced with light having a wavelength in the range of about 710 nm to about 750 nm to measured fluorescence induced with light having a wavelength in the range of about 830 nm to about 870 nm in retinal pigment epithelium cells compared to a reference ratio is indicative of increased risk of retinal degeneration.

In other embodiments, an image of detected two-photon induced fluorescence of a retina irradiated with short pulse light from a laser having a wavelength in the range of about 710 nm to about 750 nm and an image of detected two-photon induced fluorescence of a retina irradiated with short pulse light from a laser having a wavelength in the range of about 830 nm to about 870 nm can be generated and compared to determine pathological changes in the retina.

In some embodiments, measuring the fluorescence induced with light from a wavelength in the range of about 710 nm to about 750 nm and measuring the fluorescence induced with light from a wavelength in the range of about 830 nm to about 870 nm can include quantifying at least one of the amount, spatial location, or spectral properties of the measured fluorescences.

In still other embodiments, a therapeutic agent can be administered to the subject prior to irradiating the retina of the subject with short pulse light from the lasers. The measured fluorescence of the retina irradiated can be compared to assess the effect of the agent on inhibiting retinal degeneration.

### BRIEF DESCRIPTION OF THE DRAWINGS

FIGS. 1(A-B) illustrate images and plots showing time course of changes in the retina of Abca4^{-/-}Rdh8^{-/-} (Dko) mice after bright light exposure. Dko mice with an albino background at 4 wk of age were exposed to 10,000 l× light for 60 min, and then kept in the dark until evaluation. (A) TPM images obtained with 730 nm excitation before and at days 1, 3, and 11 after light exposure. Photoreceptor outer segment layer images are shown (Upper). Faint at day 1 and more easily visible at day 3 after light exposure, round AF spots (arrowheads) were observed. Large AF granules (arrow) were visualized at day 3 after light. At day 11 after illumination, AF-elongated shapes (arrows) were seen. (Scale bars: 25 μm). RPE images displaying characteristic double-nuclei structures (Lower). AF particles located close to the RPE cellcell junctions at days 1 and 3 after exposure are indicated with white arrowheads. AF particles, observed at day 11 after light exposure, are designated with yellow arrowheads. (B) At day 3 after light exposure, AF TPM emission spectra were obtained from the RPE layer, the ROS round spots, and large granules. (C) Amounts of retinyl esters (RE) and 11cRAL in the eye were quantified by HPLC on indicated days after light exposure. Error bars indicate SD of the means (n=5). *P<0.05 in RE and #P<0.05 in 11cRAL vs. values obtained in no-light-exposed mice. (D) Retinal function evaluated by ERG recordings decreased significantly at day 1 from rod (P<0.05 at all stimulus intensities by one-way ANOVA) but not cone photoreceptors (P>0.5 by one-way ANOVA at a stimulus intensity greater than −2 log cd s m−2) and then disappeared entirely from both types of photoreceptors at days 3 and 10 after light exposure. Error bars indicate SD of the means (n=3).

FIG. 2 illustrates images showing temporal changes in AF particles after light exposure. TPM images of intact Dko mouse eyes after 10,000 l× light exposure for 60 min. In each 3D TPM section, the RPE is at the top as indicated by 0 μm on the z axis and the ROS are underneath. Without light exposure, only small weak AF spots were detected in the plane located 8 μm below the RPE layer, as indicated by white arrowheads (Top). However, both round doughnut-shaped AF spots and large AF granules were located ˜8 μm beneath the RPE at day 3 after light exposure (Middle). At day 11 after light exposure, round doughnutshaped AF spots were no longer present, but predominant larger AF shapes were extending to deeper level (˜12 μm) beneath the RPE (Bottom).

FIGS. 3(A-B) illustrate images and a plot showing numbers of AF microglia/macrophages in Dko mice after light exposure. (A) Dko mice with a pigmented background at 4 wk of age were exposed to 10,000 l× light for 30 min and then kept in the dark until evaluation. Representative SLO images after light exposure are presented at days 3, 7, and 21 after light exposure (Left). Numbers of AF granules identified by SLO after light exposure were counted on the indicated day (Right). Bars indicate SD (n=5). *P<0.05 vs. no light exposure. (B) Cx3crlgfp/ΔAbca4^{-/-}Rdh8^{-/-} mice with an albino background at 4 wk of age were exposed to 10,000 l× light for 60 min Microglial translocation was examined by fluorescent imaging (Top) and by TPM imaging at indicated depths of the retina (Middle and Bottom). Microglial cells (arrowheads) in these mice exhibited GFP expression because of their Cx3crlgfp/Δ genotype. DAPI was used to stain nuclei. INL, inner nuclear layer; ONL, outer nuclear layer; OPL, outer plexiform layer; SRS, subretinal space. (Scale bars: 50 μm.)

FIGS. 4(A-B) illustrate plots showing light-induced differences in metabolic profiles of mouse retina. Analysis of light-induced differences in metabolic profiles of mouse retina. (A) (Upper) Base peak ion chromatograms for retinal extracts obtained from dark-adapted (black trace) and, at day 3 after light exposure (red trace), Dko mice. The blue line corresponds to samples obtained from retinylamine (Ret-NH2) treated animals. The same color scheme was used for chromatograms obtained from Lrat^{-/-} mice (Lower; blue line indicates Ret-NH2-treated Dko retinas). (B) The most characteristic ions overrepresented in light-exposed samples are shown. Differences between analyzed samples are represented in differential feature plots with the minimal fold-change threshold set at 1.5 and P value threshold at <0.01. In this representation, the most dominant increase in ion intensities found in light-exposed retinas vs. reference samples are shown in red, whereas suppressed ions are marked in green. The size of each circle represents the log-fold change. The shade of the color corresponds to the P value (the darker the color, the lower the P value). The most characteristic common clusters of ions for lightexposed samples are circled in yellow (see Results).

FIGS. 5(A-C) illustrate images and a graph showing Dko mice exhibit enlargement of photoreceptor cell outer segments at day 1 after light exposure. Dko mice with an albino background at 4 wk of age were exposed to 10,000 l× light for 60 min and then kept in dark until evaluation. TPM imaging was carried out at day 1 after light exposure immediately after retinas were removed from their eyecups and stripped of the RPE. Retinas for TPM were placed in 3-cm dishes with DMEM. (A) Outer segment tips are at the top, as indicated by 0 μm on the z axis. (Upper) A 3D TPM section shows regularly arranged photoreceptors in the retina from a mouse unexposed to light (Lower). A 3D TPM section reveals ROS with enlarged diameters and darker centers in a mouse retina at day 1 after light exposure. (B) Diameters and lengths of ROS from mice unexposed to light and at day 1 after light exposure are presented. *P<0.05 vs. no-light-exposed mice. (C) Magnified views of the ROS XY sections from retinas in A are shown.

FIGS. 6(A-B) illustrate images and a plot showing retinal degeneration is induced by atRAL in ex vivo retinal cultures. Retinas were removed from the eyecups of 4-wk-old C57BL/6J mice and cultured for 16 h at 37° C. Then retinas were incubated further with/without 30 μM of atRAL in the presence/absence of experimental drugs for 6 h at 37° C. Vehicle (DMSO), retinylamine (Ret-NH2) at 30 μM, or apocynin (Apo) at 300 μM was applied, together with atRAL. (A) Retinal morphology was examined after incubation with atRAL and with and without drugs. Cryosections were prepared and photoreceptor outer segments were stained with anti-rhodopsin (Rho) antibody, and nuclear staining was achieved with DAPI. The ONL was markedly disrupted in atRAL/vehicle-treated mice in contrast to either Ret-NH2- or apocynin-treated mice (Scale bars: 20 μm). (B) TUNEL staining was performed with the ApoTag Peroxidase in Situ Apoptosis Detection Kit. Counter nuclear staining was accomplished with methyl green. As in A, cotreatment with either Ret-NH2 or APO protected the ONL and INL against atRAL-induced damage (Scale bars: 20 μm). (C) An LDH assay was carried out with the LDH activity assay kit (BioVision) to calculate cell death rates in retinal culture supernatants. atRAL caused retinal cell death which was reduced by coincubation with either retinylamine or apocynin Bars indicate SD (n=3). *P<0.05. (D) Retinas were removed from eyecups of 4-wk-old C57BL/6J mice and cultured with or without 30 μM of atRAL for 24 h at 37° C. After incubation, retinas were washed twice with PBS and then examined by TPM. Spectra from photoreceptor outer segments are shown (Left) along with representative magnified images (Right). Spectra from retinas cultured with atRAL featured a broad maximum absorption at 600 nm.

FIG. 7 illustrates images showing differences in RPE AF between Dko and Mertk^{-/-}Dko mice at day 7 after light exposure. Mertk^{-/-}Abca4^{-/-}Rdh8^{-/-} (Mertk^{-/-}Dko) and Dko mice with an albino background at 3 wk of age were exposed to 10,000 l× light for 60 min, and 3D TPM images were obtained at day 7 after light exposure. Three-dimensional images of a Dko and a Mertk^{-/-}Dko mouse retina are shown in Upper and Lower, respectively. RPE in Dko exhibited an increased accumulation of AF spots, whereas no such changes were noted in Mertk^{-/-}Dko mice.

FIGS. 8(A-B) illustrate images and graphs showing differences in spectral properties of RPE fluorophores at different time points after bleaching. (A) Abca4^{-/-}Rdh8^{-/-} mice with an albino background at 4 wk of age were exposed to 10,000 l× light for 60 min, and then kept in the dark until evaluation by two-photon microscopy. Images of retinal pigmented epithelium (RPE) were obtained with 730 nm to reveal retinyl esters (RE) and with 850 nm to detect all-trans-retinal (atRAL) condensation products (1) before light, at day 1, and at day 11 after light exposure. All images were acquired with the same laser power and detector settings. Increased fluorescence from Res located in retinosomes near RPE cell borders is prominent in images obtained with 730 nm excitation at day 1 after exposure to light. The retinosomes are not distinguishable in images acquired with 850 nm excitation (Lower). At day 11 after light exposure, larger fluorescent particles indicated with yellow arrowheads emitted AF signals in response to both 730 nm and 850 nm excitation. (B) Impact of different fluorophores was quantified by plotting the ratio of 850 nm-induced fluorescence to that induced by 730 nm excitation. Bars indicate SD (n=3). *P<0.05.

FIGS. 9(A-B) illustrate images showing time course of changes in the retina of WT mice after bright light exposure. (A) Littermate WT mice of Abca4^{-/-}Rdh8^{-/-} mice with an albino background at 4 wk of age were exposed to 10,000 l× light for 60 min, and then kept in the dark until evaluation. WT mice did not develop light-induced retinal degeneration. Two-photon microscopy (TPM) images were obtained with 730 nm excitation before and at days 1, 3, and 11 after light exposure. Photoreceptor outer segment layer images are shown (Upper). No apparent changes were detected before and after light exposure (Scale bars: 25 μm). RPE images (Lower). Slightly increased autofluorescence (AF) was detected from retinosomes at day 1 and day 3 after light exposure, and returned to the level of no light exposure at day 11. (B) Retinal degeneration was induced in BALB/c mice at 4 wk of age by exposure to 20,000 l× light for 120 min. TPM images were obtained with 730 nm excitation before and at days 1, 3, and 11 after light exposure. Photoreceptor outer segment layer images (Upper). Round AF spots (red arrowheads) and large AF granules (white arrow) were detected at day 3 after light exposure. At day 11 after illumination, AF elongated shapes (magenta arrows) were seen (Scale bars: 25 μm). RPE images (Lower). AF particles located close to the RPE cellcell junctions at day 1 and day 3 after light exposure are indicated with white arrowheads. Larger AF particles observed on day 11 after light exposure are designated with yellow arrowheads.

FIGS. 10(A-C) illustrate images showing effects of pharmacological treatments on AF and retinal preservation in Abca4^{-/-}Rdh8^{-/-} mice. Abca4^{-/-}Rdh8^{-/-} mice with an albino background at 4 wk of age were pretreated with either retinylamine (Ret-NH2) at 1 mg per mouse in 100 μL soybean oil by oral gavage 16 h before light exposure at 10,000 l× for 60 min or with apocynin (Apo) at 1 mg/mouse in 50 μL DMSO injected intraperitoneally 30 min before light illumination. After exposure to light, all mice were kept in the dark until evaluation. To investigate the impact of these drugs on AF deposits as observed in FIG. 1, TPM was performed at day 7 after light exposure with 730 nm excitation to reveal RE in retinosomes and with 850 nm excitation to detect atRAL condensation products. (A) TPM imaging of RPE with 730 nm excitation (Upper) and with 850 nm excitation (Lower). The RPE of Ret-NH₂-treated mice exhibited increased fluorescence of retinosomes resulting from RPE65 inhibition (1), whereas Apo-treated mice exhibit an RPE appearance similar to mice unexposed to light. Increased numbers of larger AF particles in the RPE were observed in light-exposed, vehicle-treated mice (730 nm excitation images). These AF particles were even more pronounced in TPM images obtained with 850 nm excitation light (Scale bars: 38 μm). (B) Ratios of AF intensity obtained with 850 nm light excitation to that obtained with 730 nm light excitation were calculated (Left) and the numbers of AF particles in the RPE were counted (Right). Bars indicate SD; *P<0.05. (C) Retinal histology with toluidine blue staining was assessed at day 7 after light exposure (Scale bars: 20 μm). Histological analyses showed preserved retinal structures in drug-treated mice, whereas severe light-induced retinal degeneration was exhibited in vehicle-treated mice. These findings indicate that the presence of these AF particles in the RPE is indicative of abnormalities in atRAL metabolism in the retina, and could be used for retinal drug screening in vivo. GCL, ganglion cell layer; INL, inner nuclear layer; IPL, inner plexiform layer; IS, inner segments; ONL, outer nuclear layer; OPL, outer plexiform layer.

FIGS. 11(A-B) illustrate images showing retinal degeneration is induced by atRAL in ex vivo Dko retinal cultures. Retinas were removed from the eyecups of 4-wk-old Abca4^{-/-}Rdh8^{-/-} (Dko) mice and cultured for 16 h at 37° C. Retinas were then incubated further with/without 30 μM of atRAL in the presence/absence of experimental drugs for 6 h at 37° C. Vehicle (DMSO), retinylamine (Ret-NH2) at 30 μM, or apocynin (Apo) at 300 μM was applied together with atRAL. (A) Retinal morphology was examined after incubation with atRAL. Cryosections were prepared and photoreceptor outer segments were stained with anti-rhodopsin (Rho) (red) antibody and nuclear staining was achieved with DAPI (blue). The ONL was markedly disrupted in atRAL/vehicle-treated mice (Scale bars: 20 μm). (B) Lactate dehydrogenase (LDH) assay was carried out with the LDH activity assay kit (BioVision) to calculate cell death rates in retinal culture supernatants. atRAL caused retinal cell death, which was reduced by coincubation with either Ret-NH₂ or Apo. Bars indicate SD (n=3). *P<0.05.

FIGS. 12(A-B) illustrate images showing changes in RPE cells of Dko mice after light exposure. Dko mice with an albino background at 4 wk of age were exposed to 10,000 l× light for 60 min and then kept in the dark until evaluation. (A) An RPE flat mount was prepared 2 wk after light exposure Immunohistochemistry performed with ZO-1 antibody revealed a focal decrease in expression of zonula occludentes (ZO-1; yellow arrowheads). No changes were observed in mice not exposed to light (Scale bars: 30 μm). (BD) Using Epon embedment followed by toluidine blue staining, we examined RPE histology 2 wk after light exposure. (B and C) Crosssectional images. (D) Horizontal section image. Yellow arrows indicate staining pattern and size changes in RPE cells and the red arrowhead microglia/macrophage (mp) (Scale bars: 20 μm). (E) Sizes of RPE cells are shown. Histological analyses of plastic sections revealed increased numbers of distorted RPE cells characterized by smaller sizes than normal RPE cells. Boxes denote interquartile range, lines within boxes denote median, whiskers denote 10th and 90th percentiles, and symbols denote outliers. *P<0.05. After light exposure, vertical cross-sections (B and C) and a horizontal section (D) at the RPE level of the retina showed pathological changes of RPE cells displayed by darker staining and indicated by yellow arrows. Measurements of the RPE cells revealed a decreased size of distorted cells compared with normal cells (E).

FIGS. 13(A-C) illustrate images showing histology of Dko mouse retinas after light exposure. Dko mice with an albino background at 4 wk of age were exposed to 10,000 l× light for 60 min and then kept in the dark until evaluation. (A) Epon-prepared tissue was used to examine retinal morphology. Toluidine blue staining revealed a decreased photoreceptor cell layer (PR) at day 3, invasion of macrophages (red arrowheads) at day 7, and RPE changes (red arrows) at day 11 after light exposure (Scale bars: 20 μm). (B) Representative electron microscopic images are shown. mp, macrophage. Yellow asterisks indicate RPE cell nuclei (Scale bars: 5 μm). (C) Cryosections reveal AF changes at day 3 after light exposure. Fragmented AF signals were detected in the photoreceptor outer segments and RPE cells of retinas from light-exposed mice (Scale bars: 10 μm). ONL, outer nuclear layer; OS, outer segment.

FIGS. 14(A-C) illustrate images showing AF changes in Mertk^{-/-}Abca4^{-/-}Rdh8^{-/-} mice after bright light exposure. Mertk^{-/-}Abca4^{-/-}Rdh8^{-/-} (Mertk^{-/-}Dko) and Abca4^{-/-}Rdh8^{-/-} (Dko) mice with an albino background at 3 wk of age were either unexposed or exposed to 10,000 l× light for 60 min, and TPM imaging was performed at day 7 after light exposure. (A) Representative TPM images captured with 730 nm and 850 nm excitation at the level of the RPE are shown. In contrast to Mertk^{-/-}Dko mice, Dko mice showed increased number of large AF particles when excited with 850 nm wavelength light compared with excitation with 730 nm light. Genetic backgrounds and excitation wavelengths are listed in the figure (Scale bars: 75 μm). (B) Three-dimensional TPM images of Mertk^{-/-}Dko mouse RPE/retinas are shown both before and at day 7 after light exposure. As opposed to Dko mouse, images of Mertk^{-/-}Dko mice revealed no obvious AF changes in the RPE layer on day 7 after light exposure. However, the presence of larger AF granules, indicative of macrophages, at ˜8 μm beneath RPE, and a decreased ROS AF signaling were evident. (C) Retinal histology with toluidine blue staining either with no light exposure or on day 3 and day 7 after light exposure is shown. White arrowheads indicate macrophages (Scale bars: 20 μm). IS, inner segments; ONL, outer nuclear layer. Reduced numbers of photoreceptor nuclei with a larger proportion exhibiting chromatin condensation were observed in the ONL after light exposure. These observations indicate that AF in particles, which appear in the RPE at day 7-11 after light exposure of Dko mice, could represent phagocytized materials from dead photoreceptor cells.

FIG. 15 illustrates images showing localization and immunohistochemistry (IHC) of AF changes in Mertk^{-/-} Dko mice after bright light exposure. Mertk^{-/-}Dko mice with an albino background at 3 wk of age were exposed to 10,000 l× light for 60 min, and TPM imaging and IHC was performed at day 3 and day 7 after light exposure. TPM images of the ROS layer (Upper) (Scale bars: 40 μm) Immunostaining reveals localization of rhodopsin (red, anti-rhodopsin Ab) in Lower Left and Lower Center and microglia/macrophages (red, antiIba-1 Ab) indicated by yellow arrowheads in Lower Right. Nuclei were stained with DAPI (blue) together with staining for rhodopsin (Scale bars: 20 μm). Increased AF in the ROS layer was evident even in retinas that were unexposed to light. The presence of macrophages indicates that these cells were responsible for removing photoreceptor debris from the subretinal space.

FIGS. 16(A-F) illustrate schematic illustrations, images, and plots showing two-photon microscopy (TPM) for imaging of mouse retina and RPE. (a) TPM system layout. DC stands for group velocity dispersion precompensation; EOM—electrooptic modulator; DM6000—upright microscope; PMT—photomultiplier tube. (b) Dichroic minor (DCh) and barrier filter 680 SPET separate fluorescence and excitation light. (c) Layout of the adaptive optics system. FMK1 and FMK2 stand for fold minors on kinematic magnetic bases; L1, L2, L3 and L4—lenses; DM—deformable minor; FM1, FM2 and FM3 fold minors. (d) Left panel, RPE image in an ex vivo 1-month-old Abca4^{-/-}Rdh8^{-/-} mouse after exposure to bright light, obtained with (top image) and without (bottom image) DC; right panel, mean fluorescence measured with and without DC; error bars indicate S.D, n=3. (e) Upper row, images of the RPE in an ex vivo 3-month-old Rpe65^{-/-} mouse obtained during DM optimization: left, at the start of optimization, with DM in the neutral position; right, at the completion of the imaging session; trial represents an image obtained with non-optimal DM settings; optimal, —an image obtained with DM settings that improved image quality. Bottom row pictures the corresponding DM surfaces. (f) Quantification of image quality, m stands for mean. Scale bars represent 100 μm.

FIG. 17 illustrates twophoton images of ex vivo mouse RPE and retina obtained through the mouse eye pupil. Excitation wavelengths and genetic background are listed in each image. (a) The RPE in 3-month-old Rpe65^{-/-} mouse eye. The inset in the right bottom quarter provides a magnified view of the RPE from the area outlined with a white rectangle. (b) The RPE in 6-month-old Abca4^{-/-}Rdh8^{-/-} mouse eye. (c) The RPE in 2-month-old WT mouse eye. (d) The ganglion cell layer in 2-month-old WT mouse eye. White arrows in b and d point to the nuclei. Scale bars represent 50 μm in all panels.

FIGS. 18(A-E) illustrate images and plots showing two-photon imaging for ophthalmic drug screening. (a) Ret-NH2 protects RPE of 1-month-old Abca4^{-/-}Rdh8^{-/-} mouse from bright light induced accumulation of fluorescent granules. Representative ex vivo images obtained 7 and 14 days after bright light exposure; images obtained with a ‘through the sclera’ configuration are included for comparison. Excitation with 730 nm was used for the upper row images whereas 850 nm was employed for the lower row. (b) Individual rod photoreceptors expressing rhodopsin-GFP fusion protein are visible in photoreceptor layer of 2-month-old hrhoG/hrhoG mice. (c) Twophoton excited emission spectra from fluorescent granules in the RPE of Abca4^{-/-}Rdh8^{-/-} mouse obtained through the sclera (black) and pupil (red). Spectrum from photoreceptors in hrhoG/hrhoG mice is shown in gray. (d) Quantification of Ret-NH2 impact on accumulation of fluorescent granules in the RPE, based on images as shown in (a); ND stands for none detected; error bars indicate S.D., n=3. (e) Lower zoom image of the RPE in 6-week-old mouse not treated with Ret-NH2, showing the optic disc is displayed in upper panel. Lower panel shows a magnified view from RPE area outlined with white rectangle in the upper image. Scale bars represent 30 μm in (a, b) and lower panel of (e) and 220 μm in the upper panel of (e).

FIGS. 19(A-E) illustrate images and plots showing set-up for two-photon RPE imaging in living mice. (a) During imaging a contact lens covers mouse eye facing the objective. (b) Representative images of a pigmented 7-week-old Abca4^{-/-}Rdh8^{-/-} mouse eye obtained in vivo with 850 nm excitation 14 days after exposure to bright light, at different depths along Z-axis; a 120 μm section through the cornea, a 1608 μm section showing lens sutures, and a 2987 μm section revealing fluorescent granules in the RPE. (c) Images of the RPE in live albino 7-week-old Rpe65^{-/-} mice obtained with 730 nm and 850 nm excitation. (d) Fluorescence emission spectra from RPE of 7-week-old Abca4^{-/-}Rdh8^{-/-} mice obtained with 850 nm and 7-week-old Rpe65^{-/-} mice obtained with 730 nm excitation light in vivo. (e) Quantification of fluorescent granules. Error bars indicate S.D., n=3.

### DETAILED DESCRIPTION

For convenience, certain terms employed in the specification, examples, and appended claims are collected here. Unless defined otherwise, all technical and scientific terms used herein have the same meaning as commonly understood by one of ordinary skill in the art to which this application belongs.

The articles “a” and “an” are used herein to refer to one or to more than one (i.e., to at least one) of the grammatical object of the article. By way of example, “an element” means one element or more than one element.

The terms “comprise,” “comprising,” “include,” “including,” “have,” and “having” are used in the inclusive, open sense, meaning that additional elements may be included. The terms “such as”, “e.g.”, as used herein are non-limiting and are for illustrative purposes only. “Including” and “including but not limited to” are used interchangeably.

The term “or” as used herein should be understood to mean “and/or”, unless the context clearly indicates otherwise.

A “patient,” “subject,” or “host” may mean either a human or non-human animal, such as primates, mammals, and vertebrates.

The term “retina” refers to a region of the central nervous system with approximately 150 million neurons. It is located at the back of the eye where it rests upon a specialized epithelial tissue called retinal pigment epithelium or RPE. The retina initiates the first stage of visual processing by transducing visual stimuli in specialized neurons called “photoreceptors”. Their synaptic outputs are processed by elaborate neural networks in the retina and then transmitted to the brain. The retina has evolved two specialized classes of photoreceptors to operate under a wide range of light conditions. “Rod” photoreceptors transduce visual images under low light conditions and mediate achromatic vision. “Cone” photoreceptors transduce visual images in dim to bright light conditions and mediate both color vision and high acuity vision.

Every photoreceptor is compartmentalized into two regions called the “outer” and “inner” segment. The inner segment is the neuronal cell body containing the cell nucleus. The inner segment survives for a lifetime in the absence of retinal disease. The outer segment is the region where the light sensitive visual pigment molecules are concentrated in a dense array of stacked membrane structures. Part of the outer segment is routinely shed and regrown in a diurnal process called outer segment renewal. Shed outer segments are ingested and metabolized by RPE cells.

The term “macula” refers to the central region of the retina, which contains the fovea where visual images are processed by long slender cones in high spatial detail (“visual acuity”). “Macular degeneration” is a form of retinal neurodegeneration, which attacks the macula and destroys high acuity vision in the center of the visual field. AMD can be in a “dry form” characterized by residual lysosomal granules called lipofuscin in RPE cells, and by extracellular deposits called “drusen”. Drusen contain cellular waste products excreted by RPE cells. “Lipofuscin” and drusen can be detected clinically by ophthalmologists and quantified using fluorescence techniques. They can be the first clinical signs of macular degeneration.

Lipfuscin contains aggregations of A2E. Lipofuscin accumulates in RPE cells and poisons them by multiple known mechanisms. As RPE cells become poisoned, their biochemical activities decline and photoreceptors begin to degenerate. Extracellular drusen may further compromise RPE cells by interfering with their supply of vascular nutrients. Drusen also trigger inflammatory processes, which leads to choroidal neovascular invasions of the macula in one patient in ten who progresses to wet form AMD. Both the dry form and wet form progress to blindness.

The term “ERG” is an acronym for electroretinogram, which is the measurement of the electric field potential emitted by retinal neurons during their response to an experimentally defined light stimulus. ERG is a non-invasive measurement, which can be performed on either living subjects (human or animal) or a hemisected eye in solution that has been removed surgically from a living animal.

The term “RAL” means retinaldehyde. “Free RAL” is defined as RAL that is not bound to a visual cycle protein. The terms “trans-RAL” and “all-trans-RAL” are used interchangeably and mean all-trans-retinaldehyde.

Embodiments described herein relate to a method of determining, measuring, and/or assessing retinal degeneration and/or increased risk retinal degeneration of photoreceptors and/or the retinal pigment epithelium (RPE) cells of a subject. It was found that light-induced production of atRAL causes RPE-independent degeneration of photoreceptor cells. Active phagocytosis of affected photoreceptor cells by the RPE is required for the development of pathological changes in the RPE and RPE degeneration develops as a consequence of phagocytosis of excess atRAL condensation products accumulated primarily in rod outer segments (ROS) after light exposure.

It was further found that repetitive, dynamic imaging of atRAL and atRAL condensation products using two-photon microscopy can be used to determine the spatial localization, spectral properties, and amounts of the atRAL and atRAL condensation products as well as detect early changes in retinoid metabolism in photoreceptor cells and RPE to assess retinal degeneration and the effectiveness of treatments of the conditions associated with retinal degeneration.

In some embodiments, the method can include irradiating the retina of the subject with short pulse light from a laser having a wavelength in the range of 600 nm to 1000 nm to stimulate two-photon induced fluorescence. Two-photon induced fluorescence is detected from inner and/or outer segments of the photoreceptor cells and/or retinal pigment epithelium of the subject using a photon detector. An image of the detected fluorescence in the inner and/or outer segments of the photoreceptors and/or retinal pigment epithelium is generated. The image is then compared to a reference image to assess photoreceptor and/or retinal pigment epithelium cell death or degeneration.

The reference image can include, for example, an image of two-photon induced fluorescence of photoreceptors and/or retinal pigment epithelium of the subject obtained at an earlier time point or age of the subject, an image of two-photon induced fluorescence of photoreceptors and/or retinal pigment epithelium of retina of an apparently healthy subject, and/or an image of two-photon induced fluorescence of photoreceptors and/or retinal pigment epithelium of the subject obtained prior to and/or after administration of a therapeutic agent.

In some embodiments, an increase in the amount or spatial localization of the fluorescence of the generated image compared to the reference image can be indicative of an increased risk of photoreceptor and/or retinal pigment epithelium cell death or degeneration

In some embodiments, a three dimensional image of the photoreceptor outer segment can be generated based on the detected fluorescence to determine the shape and/or volume of the outer segment of the photoreceptor. An increase in volume of the photoreceptor outer segment compared to a reference volume of a photoreceptor can be indicative of an increased risk of photoreceptor death. The increased volume of the photoreceptor outer segment compared to the reference volume can be at least about 50%, at least about 75%, at least about 100%, at least about 150%, at least about 200%, at least about 300% or more.

In some embodiments, the light used to irradiate the retina can have a wavelength in the range of about 710 nm to about 750 nm (e.g., about 730 nm).

In other embodiments, a three dimensional image of the retinoid cycle fluorophores in the retinal pigment epithelium can be generated based on the detected fluorescence to determine the amount or spatial localization of the retinoid cycle fluorophores in the retinal pigment epithelium. The light used to irradiate the retina has a wavelength in the range of about 830 nm to about 870 nm (e.g., about 850 nm).

Other embodiments described herein relate to a method of determining retinal degeneration in a subject that includes measuring two-photon induced fluorescence of a retina irradiated with short pulse light from a laser having a wavelength in the range of about 710 nm to about 750 nm and a wavelength in the range of about 830 nm to about 870 nm. The measured fluorescence of the retina irradiated with light from a wavelength in the range of about 710 nm to about 750 nm is compared with the measured fluorescence of the retina irradiated with light from a wavelength in the range of about 830 nm to about 870 nm to assess pathological changes in the retina.

In some embodiments, an increase in the ratio of measured fluorescence induced with light from a wavelength in the range of about 710 nm to about 750 nm to measured fluorescence induced with light from a wavelength in the range of about 830 nm to about 870 nm in photoreceptor cells compared to a reference ratio is indicative of increased risk of photoreceptor cell death.

The reference ratio can include, for example, a ratio of measured fluorescence induced with light from a wavelength in the range of about 710 nm to about 750 nm to measured fluorescence induced with light from a wavelength in the range of about 830 nm to about 870 nm of photoreceptors and/or retinal pigment epithelium of the subject obtained at an earlier time point or age of the subject, of an apparently healthy subject, and/or of the subject obtained prior to and/or after administration of a therapeutic agent.

In other embodiments, decrease in the ratio of measured fluorescence induced with light from a wavelength in the range of about 710 nm to about 750 nm to measured fluorescence induced with light from a wavelength in the range of about 830 nm to about 870 nm in retinal pigment epithelium cells compared to a reference ratio is indicative of increased risk of retinal degeneration.

In other embodiments, an image of detected two-photon induced fluorescence of a retina irradiated with short pulse light from a laser having a wavelength in the range of about 710 nm to about 750 nm and an image of detected two-photon induced fluorescence of a retina irradiated with short pulse light from a laser having a wavelength in the range of about 830 nm to about 870 nm can be generated and compared to determine pathological changes in the retina.

In some embodiments, measuring the fluorescence induced with light from a wavelength in the range of about 710 nm to about 750 nm and measuring the fluorescence induced with light from a wavelength in the range of about 830 nm to about 870 nm can include quantifying at least one of the amount, spatial location, or spectral properties of the measured fluorescences.

In the practice, a portion of a mammalian retina can be irradiated, in vivo, with light having a wavelength in the range of from 600 nm to 1000 nm (e.g., from about 710 nm to about 730 nm (e.g., about 730 nm) or from about 830 nm to about 870 nm (e.g., about 850 nm)) at an intensity sufficient to stimulate two-photon-induced fluorescence within the retina. The two-photon induced fluorescence has a wavelength in the range of from 400 nm to 640 nm depending on the retinoid or retinoid condensation produce irradiated. The two-photon induced fluorescence is measured for a period of time sufficient to obtain enough information to be able to assess photoreceptor and/or retinal pigment epithelium cell death and/or degeneration.

The retina can be irradiated over an area of from 250 μm² to 500,000 μm², or a larger or smaller area of the retina may be irradiated with laser light. Typically, irradiation of a larger area of the retina (e.g., greater than about 1000 μm²) is done by irradiating the retina through the pupil, as described more fully herein. More than one area of the retina may be irradiated with laser light.

The intensity of the irradiating light is selected to generate sufficient photon flux at the area where the beam of light impinges on the retina so that there is a high chance of two photons being simultaneously absorbed by a molecule capable of fluorescence (e.g., retinyl ester). The intensity of the irradiating light should not be so great that it causes a significant amount of cellular damage. Thus, the optical power of the irradiating light, at a fixed focal volume of the retina, is typically in the range of from 0.05 mW to 25 mW, such as from 0.5 mW to 15 mW. Scanning the laser light across the retina allows higher optical powers to be used.

In some embodiments of the methods of the present invention, the retina is illuminated through the sclera. The sclera can significantly scatter the illuminating light passing there through, and so, when anatomically feasible, the retina is typically illuminated at the thinnest point of the sclera. For example, the thinnest region of the human sclera is at the equatorial region located around the circumference of the eye approximately midway between the pupil and the portion of the retina located directly opposite the pupil.

The laser can be a component of a laser scanning microscope or, for example, a component of a scanning laser ophthalmoscope. By way of example, a commercially available laser scanning microscope can be modified to illuminate the retina of a mammalian eye. Examples of commercially available laser scanning microscopes that can be modified to illuminate the retina of a mammalian eye include a Leica TCS SP5 (Leica Microsystems Inc., Bannockburn, Ill., U.S.A.).

Modifications to adapt a commercially available laser scanning microscope for use in the method described herein include physically turning the microscope tube and final objective lens from a vertical orientation to a horizontal orientation. Pre-conditioning of the near infra red laser beam may be necessary to counteract the temporal pulse broadening arising from the modified laser scanning microscope optical system and human tissue. An eye-cup may be used to hold index matching liquid (e.g., oil) or gel between the objective lens and the sclera. An objective lens may be used that has a reduced outer diameter at the distal end so that it can more easily reach the equatorial region of the human sclera when the mammalian subject looks far askance.

The microscope may be modified to include one or more photon counting modules for the optical detection of fluorescence, and possibly photons resulting from second harmonic generation.

The laser can have a repetition frequency in the range of, for example, from 76 MHz to 100 MHz. With appropriate modification, the laser can have a repetition frequency in the range of from 1 kHz to 250 kHz.

The laser can have a pulse length in the range of, for example, from 10 femtoseconds to 1000 fs, such as from 35 fs to 200 fs. The laser light can be scanned over a portion of a mammalian retina (e.g., scanned vertically, and/or scanned horizontally, and/or scanned in a regular and/or irregular geometric pattern), or directed onto a defined area of the retina without scanning Thus, for example, the light pulse frequency may be from 1 pulse to 500 pulses per imaging pixel when the light is scanned onto the retina, and at least 500 pulses per imaging pixel when the irradiating beam is stationary, or substantially stationary.

By way of example, a Leica (Wetzlar, Germany) TCS SP5 can be modified to include: an upright DM600 microscope stand, a Chameleon VisionS (Coherent, Santa Clara, Calif.) femtosecond laser, an objective with a 0.5 numerical aperture and 15 mm working distance, and a custom adaptive optics system including a deformable mirror (DM).

In some embodiments, the laser can be directed to a deformable mirror prior to irradiating a focal volume of the retina. The deformable mirror can provide fine focus adjustment and aberration correction of the laser on focal volume of the retina. The shape of the deformable mirror can be controlled by an image quality metric feedback without the use of a wavefront sensor. A plurality of Zernike nodes can be used as basis functions for deformation of the deformable mirror and focus and excitation of the laser. In some embodiments, the Zernike nodes can be sequentially optimized or optimized using a stochastic parallel gradient descent method.

The described methods can also be used for screening or determining the therapeutic effect, toxicity, or clinical outcome of agents or drugs in inhibiting photoreceptor and/or retinal pigment epithelium cell death or degeneration. For examples, the methods can include administering a therapeutic agent to the subject prior to irradiating the retina of the subject with short pulse light from the laser, and comparing the image to a reference image to assess the effect of the compound on inhibiting photoreceptor cell and/or retinal pigment epithelium death or degeneration.

The subject can be, for example, human or a genetically engineered animal. In one example, the genetically engineered animal is a genetically engineered Abca^{-/-}Rdh8^{-/-} mouse.

In some embodiments, the retina of the subject can be irradiated with light effective to induce retinal degeneration prior to irradiating the retina to stimulate two photon induced fluorescence. For example, the retina of the subject can be photo-bleached prior to irradiating the retina to stimulate two photon induced fluorescence.

In certain embodiments, the methods described herein can be used to determine an optimal dose of an agent or drug for administration to a subject (e.g., a dose that provides an optimal therapeutic effect and/or minimal toxicity effect when administered to a subject). In some embodiments, the methods described herein can be used for screening a drug at two, three or more dosages (e.g., predicting the therapeutic effects and/or toxicity effects of two, three or more dosages of a test drug), and selecting the dosage that is predicted to achieve a therapeutic effect and/or predicted to cause minimal or no toxicity (e.g., minimal or no serious side effects). In some embodiments, a reference database is generated using the methods described herein of the effects on molecular change in retinoid metabolism of a reference drug administered at two, three or more dosages (such as a medium dosage, a low dosage, and/or a high dosage; or a therapeutically effective dosage, a dosage that is not therapeutically effective, and/or a dosage that is known to cause one or more side effects)

Any agent, compound, or drug known in the art or later discovered can be utilized (e.g., as a test compound or as a reference compound) in accordance with the methods described herein including, without limitation, small molecules and biological molecules, such as cells, antibodies, proteins, peptides, antisense, DNA or RNA, and RNAi.

In some embodiments, the agent is a reference compound that has been shown to produce a therapeutic effect and/or has been characterized for toxicity in clinical studies in a non-human animal or in a human (preferably, human clinical studies). In some embodiments, the agent is a test compound, e.g., a compound whose therapeutic efficacy or toxicity characteristics are not known. In specific embodiments, the agent is a test compound the therapeutic efficacy and/or toxicity characteristics of which it is desirable to predict and/or determine. In certain embodiments, the test compound is an analog or derivative of one or more reference compounds (e.g., 2, 3, 4, 5, or more than 5 compounds, or a mixture of compounds) that have known therapeutic and/or toxicity effects (e.g., for testing whether the test compound has clinical benefits in comparison to the reference compound(s) such as improved therapeutic or toxicity characteristics). In some embodiments, more than one test compound is used in the methods described herein (e.g., 2, 3, 4, 5, 6, 7, 8, 9, 10 or more than 10 compounds). In certain embodiments, the test compound is a mixture of two, three or more compounds. In other embodiments, the test compound is a single compound—not a mixture of compounds.

In some embodiments, the agent can include at least one of a Gs or Gq coupled serotonin receptor antagonist, such as 5-HT₂ₐ receptor antagonists, 5-HT_{2b} receptor antagonists, 5-HT₂ receptor antagonists, 5-HT_{2a/c} receptor antagonists, 5-HT₄ receptor antagonists, 5-HT₆ receptor antagonists, and 5-HT₇ receptor antagonists, an alpha 1 adrenergic antagonist, an alpha-2 adrenergic receptor agonist, and adenylyl cyclase inhibitor, an M3 receptor antagonist, a PLC inhibitor, or a primary amine, which forms transient shiff-bases with all-trans retinal in the eye.

Examples of serotonin receptor antagonists are citalopram, escitalopram, fluoxetine, R-fluoxetine, sertraline, paroxetine, fluvoxamine, venlafaxine, duloxetine, dapoxetine, nefazodone, imipramine, imipramine N-oxide, desipramine, pirandamine, dazepinil, nefopam, befuraline, fezolamine, femoxetine, clomipramine, cianoimipramine, litoxetine, cericlamine, seproxetine, WY 27587, WY 27866, imeldine, ifoxetine, tiflucarbine, viqualine, milnacipran, bazinaprine, YM 922, S 33005, F 98214-TA, OPC 14523, alaproclate, cyanodothepine, trimipramine, quinupramine, dothiepin, amoxapine, nitroxazepine, McN 5652, McN 5707, O1 77, Org 6582, Org 6997, Org 6906, amitriptyline, amitriptyline N-oxide, nortriptyline, CL 255.663, pirlindole, indatraline, LY 113.821, LY 214.281, CGP 6085 A, RU 25.591, napamezole, diclofensine, trazodone, EMD 68.843, BMY 42.569, NS 2389, sercloremine, nitroquipazine, ademethionine, sibutramine, clovoxamine, desmethylsubitramine, didesmethylsubitramine, clovoxamine vilazodone, N-[(1-[(6-Fluoro-2-napthalenyl)methyl]-4-piperidinyl]amino]carbonyl]-3-pyridine carboxamide, [trans-6-(2-chlorophenyl)-1,2,3,5,6,10b-hexahydropyrrolo-(2,1-a)isoquinol-ine] (McN 5707), (dl-4-exo-amino-8-chloro-benzo-(b)-bicyclo [3.3.1] nona-2-6 alpha (10 alpha)-diene hydrochloride) (Org 6997), (dl)-(5 alpha,8 alpha,9 alpha)-5,8,9,10-Tetrahydro-5,9-methanobenzocycloocten-8-amine hydrochloride (Org 6906), -[2-[4[(6-fluoro-1H-indol-3-yl)-3,6-dihydro-1(2H)-pyridinyl]ethyl]-3-isop-ropyl-6-(methylsulphonyl)-3,4-dihydro-1H-2,1,3-benzothiadiazine-2,2-dioxid-e (LY393558), [4-(5,6-dimethyl-2-benzofuranyl)-piperidine] (CGP 6085), dimethyl-[5-(4-nitro-phenoxy)-6,7,8,9-tetrahydro-5H-benzocyclohepten-7-yl-]amine (RU 25.591), or a pharmaceutically acceptable salt of any of these compounds.

In one embodiment, the serotonin receptor antagonist is selected from agomelatine, pizotifen, RS 23579-190, Ro 04-6790 (4-Amino-N-[2,6-bis(methylamino)-4-pyrimidinyl]benzenesulfonamidev), SGS 518 oxalate (1-methyl-3-(1-methyl-4-piperidyl)indol-5-yl]2,6-difluorobenzenesulfonate; oxalic acid), SB 269970 (3-({(2R)-2-[2-(4-Methyl-1-piperidinyl)ethyl]-1-pyrrolidinyl}sulfonyl)phenol hydrochloride (1:1)), LY 215840 ((8β)-N-[(1S,2R)-2-Hydroxycyclopentyl]-1-isopropyl-6-methylergoline-8-carboxamide), citalopram, escitalopram, fluoxetine, sertraline, paroxetine, fluvoxamine, venlafaxine, duloxetine, dapoxetine, nefazodone, imipramine, femoxetine and clomipramine or a pharmaceutically acceptable salt of any of these compounds.

Examples of 5-HT₂ₐ receptor antagonists are described in U.S. Pat. No. 4,444,778 and can include nefazodone, pizotifen, ketanserin, desipramine, imipramine, chlorimipramine, protriptylene, dibenzepine, amitryptyline, doxepin, prothiadene, pirandamine, spirobenzofuran, ciclazindol, nefopam, deximafen, daledalin, amedalin, quipazine, trazodone, zimelidine, tofenacine, fenetazole and fenflurame. Additional compounds which have 5-HT₂ₐ antagonist activity and can be used are 11-amino-1,5-methano-1,2,5,6-tetrahydrobenzocine; 1-methylamino-4-phenyl-1,2,3,4-tetrahydronaphthylene; 6-cyano-1,3-dihydro-3-dimethylaminopropyl-3-(p-fluorophenyl)-isobenzofuran; 4-benzyl-1-(2-benzofurancarbonyl)-piperidide, 1,4-ethano-4-phenyl-cyclohexylamine, α-(p-chlorophenyl)-2-methylaminomethylbenzyl alcohol; α-(2-methylaminoethyl)-2-methoxy or 4-trifluoromethylphenylbenzyl ether or p-anisyl-(1-methyl-4-phenyl-3-pipecolinyl)-ether. Still other examples of 5-HT₂ₐ receptor antagonists include piperidinylamino-thieno[2,3-d]pyrimidine compounds described in U.S. Pat. No. 7,030,240 and 1,4-substituted cyclic amine derivatives described in U.S. Pat. No. 7,541,371

Examples of alpha 1 adrenergic receptor antagonists that can include phentolamine family antagonists, known as imidazolines, alkylating agents such as phenoxybenzamine, or piperazinyl quinazolines.

In specific embodiments, the alpha 1 adrenergic receptor antagonist can include, for example, doxazosin, prazosin, tamsulosin, terazosin and 5-methylurapadil. The syntheses of these compounds are described in U.S. Pat. Nos. 3,511,836, 3,957,786, 4,026,894, 5,798,362, 5,792,767, 5,891,882, 5,959,108, and 6,046,207. Additionally, other alpha 1 adrenergic receptor antagonist are well known in the art. See, for example, Lagu, “Identification of alpha 1A-adrenoceptor selective antagonists for the treatment of benign prostatic hyperplasia”, Drugs of the Future 2001, 25(8), 757-765 and Forray et al., 8 Exp. Opin. Invest. Drugs 2073 (1999), hereby incorporated by reference in its entirety, which provide examples of numerous alpha 1 adrenergic receptor antagonists.

Examples of alpha-2 adrenergic receptor agonists include L-norepinephrine, clonidine, dexmetdetomidine, apraclonidine, methyldopa, tizanidine, brimonidine, xylometazoline, tetrahydrozoline, oxymetazoline, guanfacine, guanabenz, guanoxabenz, guanethidine, xylazine, medetomide, moxonidine, mivazerol, rilmenidine, UK 14,304, B-HT 933, B-HT 920, octopamine or a combination thereof.

Other examples of alpha-2 adrenergic receptor agonists include, but are not limited to amidephrine, amitraz, anisodamine, apraclonidine, cirazoline, detomidine, epinephrine, ergotamine, etilefrine, indanidine, lofexidine, medetomidine, mephentermine, metaraminol, methoxamine, midodrine, naphazoline, norepinephrine, norfenefrine, octopamine, oxymetazoline, phenylpropanolamine, rilmenidine, romifidine, synephrine, talipexole, tizanidine, or a combination thereof.

Examples of adenylyl cyclase inhibitors are 9-tetrahydrofuryl adenine, such as THFA or SQ 22536, 2′,5′-dideoxyadenosine, or 9-(cyclopentyl)-adenine.

Examples of M3 receptor antagonists include 4-DAMP or tolterodine. Other examples of M3 receptor antagonists are described in U.S. Pat. Nos. 7,723,356, 7,361,648, and 7,947,730.

Examples of PLC inhibitors are described in U.S. Pat. No. 6,235,729 and can include U73122 (1-(6-((17β-3-methoxyestra-1,3,5(10)-trien-17-yl)amino)hexyl)-1H-pyrrole-2,5-dione), ET-18-OCH₃ (1-O-octadecyl-2-O-methyl-sn-glycero-3-phosphorylcholine), and RHC-80267 (1,6-bis-(cyclohexyloximinocarbonylamino)-hexane). Still other examples of PLC inhibitors can include a-hydroxyphosphonate compounds described in U.S. Pat. No. 5,519,163.

In some embodiments, the agents used in methods described herein can be administered to the subject to treat the ocular disorder (e.g., macular degeneration, geographic atrophy, diabetic retinopathy, retinitis pigmentosa, or Stargardt disease) using standard delivery methods including, for example, ophthalmic, topical, parenteral, subcutaneous, intravenous, intraarticular, intrathecal, intramuscular, intraperitoneal, intradermal injections, or by transdermal, buccal, oromucosal, oral routes or via inhalation. The particular approach and dosage used for a particular subject depends on several factors including, for example, the general health, weight, and age of the subject. Based on factors such as these, a medical practitioner can select an appropriate approach to treatment.

Treatment according to the method described herein can be altered, stopped, or re-initiated in a subject depending on the status of ocular disorder determined by the methods described herein. Treatment can be carried out as intervals determined to be appropriate by those skilled in the art. For example, the administration can be carried out 1, 2, 3, or 4 times a day. In another embodiment, the primary amine compound can be administered after induction of macular degeneration has occurred.

The treatment methods can include administering to the subject a therapeutically effective amount of the agents alone or in combination. Determination of a therapeutically effective amount is within the capability of those skilled in the art. The exact formulation, route of administration, and dosage can be chosen by the individual physician in view of the subject's condition.

In some embodiments, the subject may be monitored for the extent of retinal degeneration using the methods described herein. Monitoring can be performed at a variety of times. For example, a subject may be monitored after a compound is administered. The monitoring can occur, for example, one day, one week, two weeks, one month, two months, six months, one year, two years, five years, or any other time period after the first administration of a compound. A subject can be repeatedly monitored using the methods described herein. In some embodiments, the dose of a compound may be altered in response to monitoring.

The invention is further illustrated by the following examples, which are not intended to limit the scope of the claims.

### Example 1

In this Example, we show that light-induced production of atRAL in Abca4^{-/-}Rdh8^{-/-} mice causes RPE-independent degeneration of photoreceptor cells. Moreover, we show that active phagocytosis of affected photoreceptor cells by the RPE is required for the development of pathological changes in the RPE. Taken together, these results support a model whereby the primary site of pathology is photoreceptor cells, with RPE degeneration developing as a consequence of phagocytosis of excess atRAL condensation products accumulated primarily in rod outer segments (ROS) after light exposure.

### Materials and Methods

### Animals

Abca4^{-/-}Rdh8^{-/-} mice were generated and all mice were genotyped by well-established methods. Mertk^{-/-} and Cx3crlgfp/A mice were purchased from The Jackson Laboratory. Mertk^{-/-}Abca4^{-/-}Rdh8^{-/-} and Cx3crlgfp/AAbca4^{-/-}Rdh8^{-/-} mice were generated by cross-breeding and then genotyped. Lrat^{-/-} mice were bred and genotyped. Only Rd8 mutation free mice with the Leu variation at amino acid 450 of RPE65 were used. Either pigmented C57BL/6J or albino C57BL/6J (C57BL/6JTyrc-2J/J) mice from The Jackson Laboratory and their littermates were used as WT controls. BALB/c mice were obtained from The Jackson Laboratory. All mice were housed in the animal facility at the School of Medicine, Case Western Reserve University, where they were maintained on a normal mouse chow diet either under complete darkness or in a 12-h light (˜10 l×)/12-h dark cyclic environment. Manipulations with retinas and retinoid extractions were done in the dark under dim red light transmitted through a Kodak No. 1 safelight filter (transmittance >560 nm). All animal procedures and experiments were approved by the Case Western Reserve University Animal Care Committees and conformed to both the recommendations of the American Veterinary Medical Association Panel on Euthanasia and the Association of Research for Vision and Ophthalmology.

### Chemicals

AtRAL, ROL, and apocynin were purchased from Sigma-Aldrich; a mixture of 0.5% tropic amide and 0.5% phenylephrine hydrochloride (Midorin-P) was obtained from Santen Pharmaceutical Co. Ltd.; xylazine/AnaSed was from LLOYD, Inc.; and ketamine/Ketaset CIII was from Fort Dodge Animal Health. Retinylamine was synthesized from retinal as previously detailed. Induction of Retinal Light Damage. Mice were dark-adapted for 12-48 h before exposure to bright light. Acute retinal damage was induced by exposing animals to 10,000 l× of diffuse white fluorescent light for either 30 min (pigmented mice) or 60 min (albino mice). For BALB/c mice, 20,000 l× for 120 min were used to induce retinal damage with EcoSmart 42 W, color temperature 2,700 K, 2,800 lumens, model 28942BD bulbs (Commercial Electric). The bulb irradiance spectrum was recorded with a calibrated spectroradiometer Specbos 1211 UV (JETI Technische Instrumente GmbH). The resulting bulb spectrum had maxima at 620, 550, 450, 405, and 340 nm, with normalized amplitudes of 1, 0.7, 0.49, 0.28, and 0.13, respectively. Before each exposure, mouse pupils were dilated with a mixture of 0.5% tropicamide and 0.5% phenylephrine hydrochloride. After light exposure, animals were kept in the dark until evaluation.

### TPM Imaging

TMP images were obtained with a Leica TCS SP5 confocal MP system equipped with an upright DM6000 CFS stand. A tunable laser Vision S (Coherent) delivered 75-fs laser light pulses at an 80-MHz pulse repetition frequency. Pulse duration at the sample was minimized by using a dispersion compensation system with settings that produced the largest two-photon excited fluorescence for the same laser power. Laser power at the sample was maintained at 3-11 mW with an electrooptic modulator. Laser light was focused on the sample with a 20×1.0 N.A. water-immersion Leica objective. Two-photon excited fluorescence was collected by the same lens and, after filtering excitation light by a Chroma ET680sp filter (Chroma Technology Corp.), the beam was directed to either PMT or HyD detectors in a nondescanned manner or to a Leica HyD detector in the descanned configuration. Emission spectra were obtained with TCS SP5 spectrally sensitive HyD detector in a descanned configuration. For imaging the RPE and retina in the intact, enucleated mouse eye, both the laser light and the resulting fluorescence had to penetrate through the sclera. Before eye enucleation, mice were anesthetized by i.p. injection of 20 μL/g body weight of 6 mg/mL ketamine and 0.44 mg/mL xylazine diluted with 10 mM sodium phosphate, pH 7.2, containing 100 mM NaCl and then euthanized in compliance with American Veterinary Medical Association Guidelines on Euthanasia, and approval by the Case Western Reserve University Institutional Animal Care and Use Committee. TPM 3D reconstructions and pixel gray values of raw retinal images were analyzed offline with Leica LAS AF 3.0.0. Sigma Plot 11.0 software (Systat Software, Inc.) was used for statistical analyses.

### ERG Recordings

All ERG experimental procedures were performed under dim red light transmitted through a Kodak No. 1 safelight filter (transmittance >560 nm) as previously described. Briefly, mice were initially dark-adapted overnight before recording; they were then anesthetized under a safety light by i.p. injection of 20 μL/g body weight of 6 mg/mL ketamine and 0.44 mg/mL xylazine diluted with 10 mM sodium phosphate, pH 7.2, containing 100 mM NaCl. Pupils were dilated with a mixture of 0.5% tropicamide and 0.5% phenylephrine hydrochloride. A contact lens electrode was placed on the eye, and a reference electrode and ground electrode were positioned on the ear and tail, respectively. ERGs were recorded by the universal testing and electrophysiological system with BigShot Ganzfeld (LKC Technologies). Single-flash recording was performed. White-light flash stimuli were used over a range of intensities (from 3.7 to 1.6 log cd·s·m−2), and flash durations were adjusted according to intensity (from 20 μs to 1 ms). Two to five recordings were made at sufficient intervals between flash stimuli (from 3 s to 1 min) to allow mice time to recover.

### Retinoid Analyses

Retinoid extraction, derivatization, and separation by HPLC were performed on eye samples from dark-adapted mice as previously described. Briefly, eyes were homogenized in 1 mL of retinoid analysis buffer [50 mM Mops, 10 mM NH₂OH, and 50% (vol/vol) ethanol in 50% (vol/vol) H₂O (pH 7.0)]. Retinoids were extracted twice with 4 mL of hexane. Then the extracted retinoids in the organic solvent were dried down in a SpeedVac The retinoids were resuspended in 0.3 mL of hexane and separated by normal-phase HPLC (Ultrasphere-Si, 4.6 μm 3×250 mm; Beckman Coulter) with 10% ethyl acetate and 90% hexane at a flow rate of 1.4 mL/min.

### Scanning Laser Ophthalmoscopy

SLO imaging was done with an HRAII instrument (Heidelberg Engineering). Mice were anesthetized by i.p. injection of a mixture (20 μL/g body weight) containing ketamine (6 mg/mL) and xylazine (0.44 mg/mL) in 10 mM sodium phosphate, pH 7.2, with 100 mM NaCl. Pupils were dilated with a mixture of 0.5% tropicamide and 0.5% phenylephrine hydrochloride before the procedure. The number of AF particles were counted per image.

### Histological Analyses

All procedures used for sample preparation, immunohistochemistry, and light microscopy were performed by well-established methods published previously. Mouse anti-rhodopsin 1D4 antibody (1:100; a gift from Robert Molday, University of British Columbia, Vancouver) and mouse anti-ZO-1 antibody (Invitrogen) were used for immunostaining. TUNEL staining was carried out with an ApoTag Peroxidase in Situ Apoptosis Detection Kit (Chemicon). Electron microscopic analyses were performed as previously described.

### Retinal Tissue Cultures

Eyes were enucleated, washed with a penicillin-streptomycin solution (Sigma), and rinsed with Hank's balanced salt solution (HyClone). Prepared mouse eyecups were flattened by creating retinal flaps. Flattened retinas were transferred onto filter paper and the retina was gently peeled off from the RPE/choroid. All these procedures were performed under a surgical microscope. Each retina on filter paper was placed into a well of a 12-well plate filled with 0.5 mL of DMEM (HyClone) with 10% FBS and incubated for 16 h at 37° C. Retinas then were washed twice with 0.5 mL of fresh DMEM containing 10% FBS and finally incubated again with/without 30 μM of atRAL for 6 h at 37° C. A lactate dehydrogenase (LDH) assay was performed to determine cellular death rates with a LDH activity assay kit (BioVision). The percentage of cytotoxicity was calculated as [(a retina with atRAL—a retina without atRAL)/(lysis control—a retina without atRAL)]×100.

### MS Analyses of Mouse Retina

At day 3 after light exposure, mouse retinas were dissected and homogenized in 0.3 mL of ice-cold acetonitrile. Samples were vortexed for 30 s followed by centrifugation for 15 min at 16,000×g. Clear supernatants were collected and used directly for LC/MS analyses. Each retinal extract was injected onto a reverse-phase C18 Phenomenex HPLC column (250×4.60 mm; 5 μm) preequilibrated with 5% acetonitrile in water. Chemical components of the retina were eluted in a linear gradient of acetonitrile from 5% to 100% (vol/vol) developed within 50 min at flow rate of 0.7 mL/min and directed onto L×Q linear ion trap MS spectrometer (Thermo Scientific) via an electrospray ionization interface operated in the positive ionization mode. Parameters for both chemical ionization and the instrument were optimized for retinal condensation products such as A2E. All solvents contained 0.1% formic acid. Total ion chromatograms were analyzed with XCMS software available online at the Scripps Center for Metabolomics.

### Statistical Analyses

Data representing the means±SD for the results of at least three independent experiments were compared by one-way ANOVA with P<0.05 considered statistically significant.

### Results

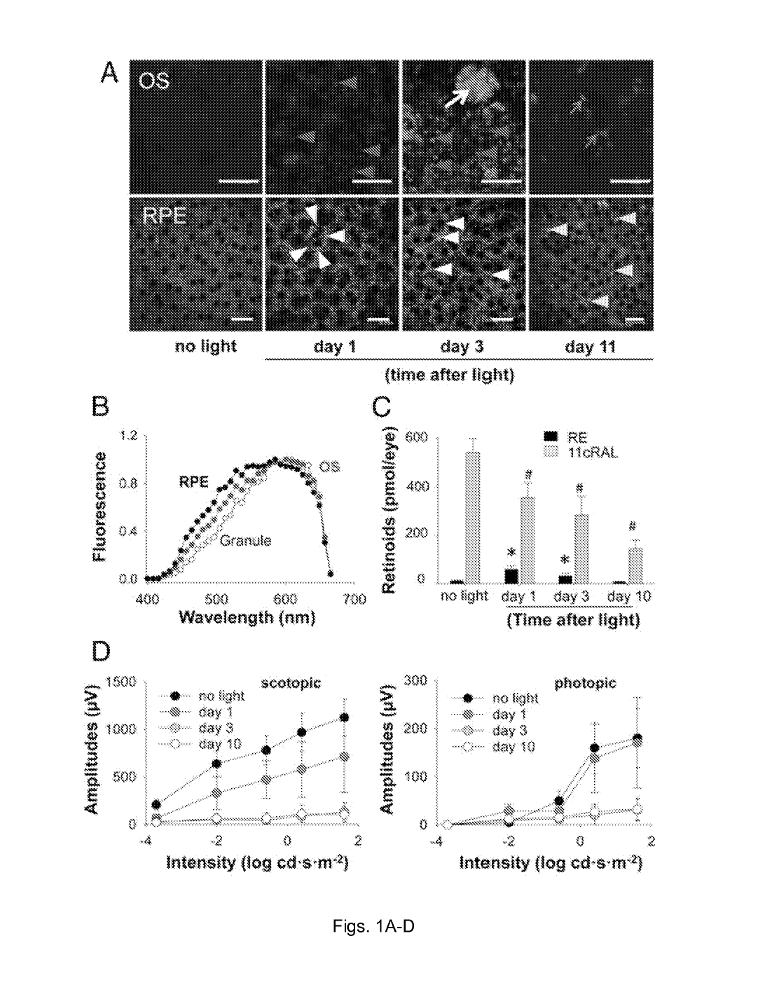

TPM noninvasively images autofluorescence (AF) signals from retinosomes containing all-trans retinyl esters (RE) and atRAL condensation products in RPE cells. As previously reported, retinosomes and other AF signals were observed in RPE cells of albino 4-wk-old Abca4^{-/-}Rdh8^{-/-} mice (32) (FIG. 1A). Additionally, we observed AF signals from photoreceptor outer segments (OS) (FIG. 1A), indicating that these AF products could be formed in the OS and then possibly be transferred to the RPE through phagocytosis. Thus, we aimed to identify the origin and site of formation of these AF products through extensive kinetic analyses of light-induced pathology in Abca4^{-/-}Rdh8^{-/-} mice by several complementary techniques, including genetic manipulations, TPM, ERGs, and liquid chromatography/mass spectrometry (LC/MS).

Characterization of Retinal AF and Function in Abca4^{-/-}Rdh8^{-/-} Mice after Bright Light Exposure

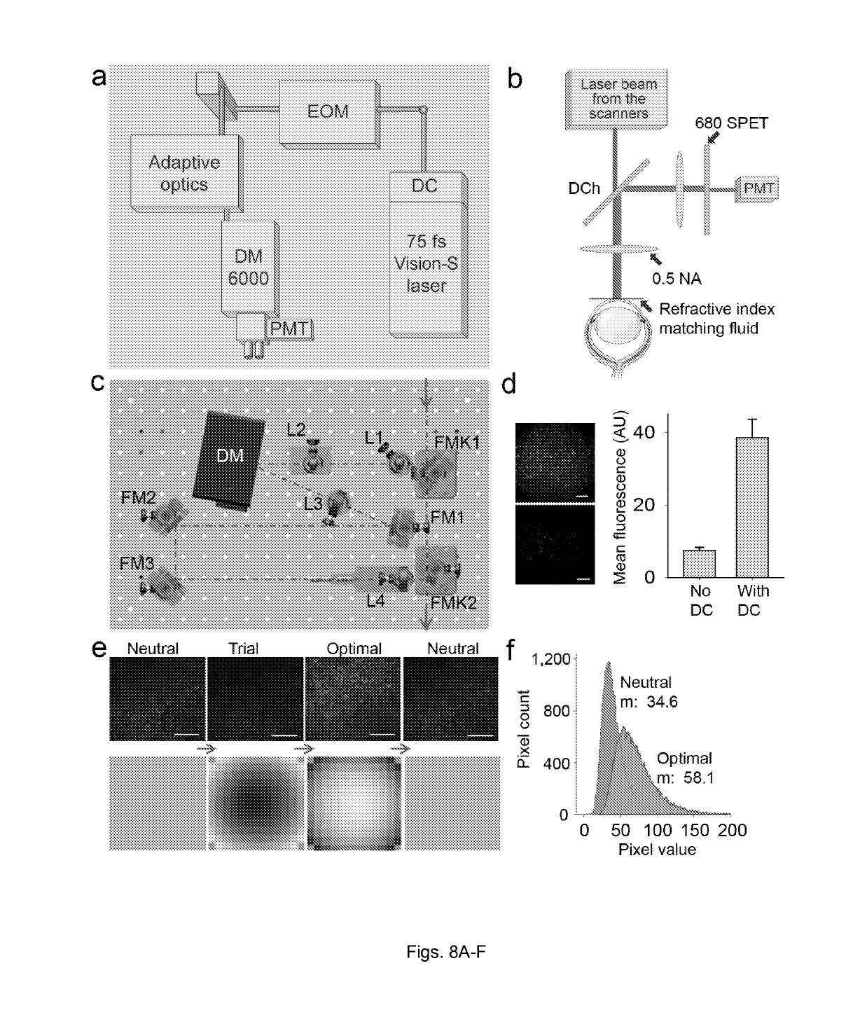

To monitor temporal changes in AF properties of OS and RPE, we examined albino 4-wk-old Abca4^{-/-}Rdh8^{-/-} mice at different time intervals after a 60-min exposure to light at 10,000 l×. Using TPM of intact mouse eyes, we observed an abundance of small AF spots in the OS at days 1 and 3 after light exposure (FIG. 1A, Upper). Moreover, ˜10× larger AF granules were detected in the OS layer at day 3 after light exposure as well. At day 11 after light exposure, small rounded AF spots, most likely representing OS, were no longer visible. Furthermore, some infiltrating cells with more elongated shapes, likely representing microglia/macrophages, were noted in the subretinal space (FIG. 1A, Upper). In parallel with the OS changes, we also noted that the intensity of AF particles located in the RPE near cell boundaries increased at days 1 and 3 after light exposure (FIG. 1A, Lower). These AF particles were clearly visible with 730 nm, but not with 850 nm, light (FIG. 1A and FIG. 8A). At day 11 after exposure, signals from these AF particles had decreased, but different types of AF signals from larger particles distributed randomly within RPE had appeared (FIG. 1A, Lower). These new AF particles were also clearly visible with 850 nm excitation at day 11 after light exposure (FIG. 8A). We have used the ratio between fluorescence recorded with excitation light at 850 nm and 730 nm to quantify changes in the fluorescent properties of these two AF signals over time (FIG. 8B).

To further characterize the origin of observed AF signals in OS and RPE, we analyzed the emission spectrum of AF by TPM of intact eyes of albino 4-wk-old Abca4^{-/-}Rdh8^{-/-} mice at day 3 after 60-min light exposure at 10,000 l×. AF spectra from the small fluorescent spots in OS and RPE showed similar patterns (FIG. 1B), except the OS spectra revealed a greater impact of fluorophores emitting at longer wavelengths with a broad maximum at 600 nm AF emission spectra from the larger granules attributed to infiltrating microglia/macrophages more closely resembled those from the OS than from the RPE, suggesting that these granules were loaded with AF debris from dying photoreceptor cells.

Because the amount of 11cRAL in the retina correlates well with the numbers of photoreceptors and can be used to quantify the severity of retinal degeneration, we used HPLC to analyze retinoids in the eye. Here we found that 11cRAL content in Abca4^{-/-}Rdh8^{-/-} mouse eyes had decreased by 37.9% at day 1 after light exposure and by 73.4% at day 10 (FIG. 1C). In contrast, RE content increased at days 1 and 3 and then decreased at day 10 after light illumination. This change in RE content also correlated well with the increase in AF elicited from particles observed in the RPE at days 1 and 3 after light exposure, and likely was due to retinosome expansion. The prolonged elevated presence of RE could be a result of compromised function of the retinoid cycle caused by the early demise of photoreceptors as indicated by the appearance of fluorescent products in the rod outer segments and further implied by the reduced quantity of 11cRAL.

Retinal function assessed by ERG recordings showed decreased responses (FIG. 1D). These ERG results indicate that substantial rod cell demise had occurred without detectible loss in cone function by day 1 after bright light exposure. Moreover, ERG signals were not detected in either rods or cones at day 3 and 10 after such exposure. These findings in Abca4^{-/-}Rdh8^{-/-} mice are consistent with progressive degeneration of both types of photoreceptor cells with greater resistance exhibited by cones to light-induced damage.

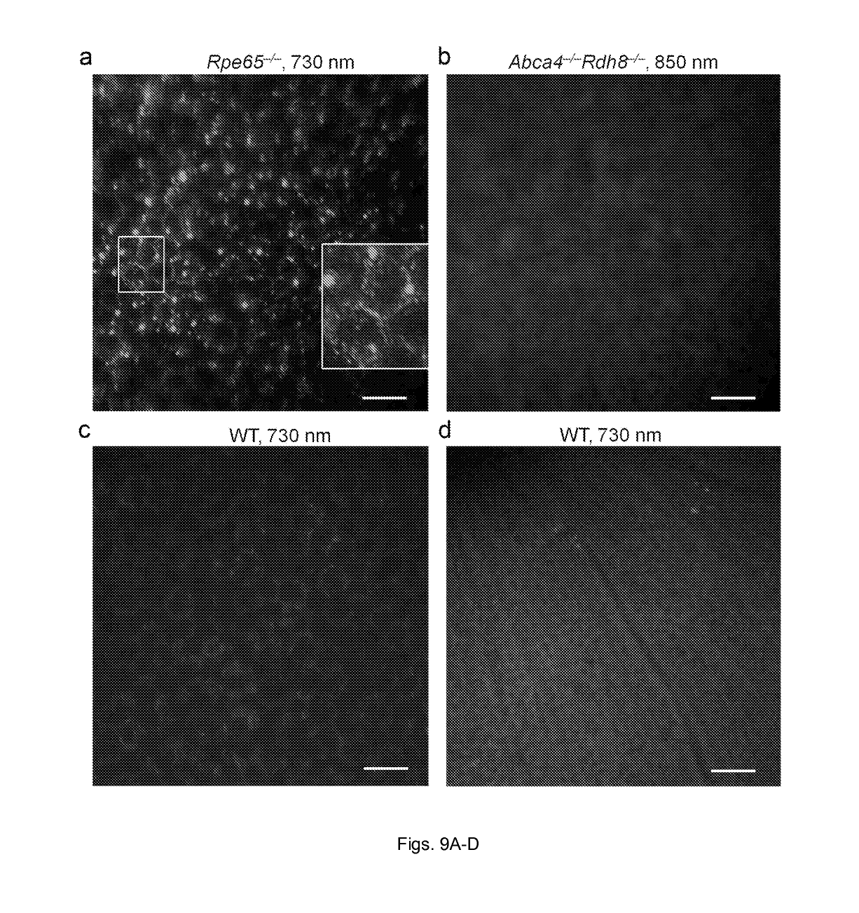

We further assessed the changes in WT mice. Littermate control WT mice of Abca4^{-/-}Rdh8^{-/-} mice were not light insensitive and did not show light-induced retinal degeneration under the same light exposure conditions as studies with Abca4^{-/-}Rdh8^{-/-} mice. As expected, no abnormal AF signals were detected by TPM imaging (FIG. 9A). Next, BALB/c mice were evaluated. Mice were exposed to 20,000 l× for 120 min to cause light-induced retinal degeneration. Damaged OS and RPE displayed AF abnormalities similar to those observed in Abca4^{-/-}Rdh8^{-/-} mice (FIG. 9B). These observations indicate that AF changes in OS and RPE are closely associated with retinal damage.

### Three-Dimensional TPM Images Reveal the Shape and Distribution of AF Signals in the Retina

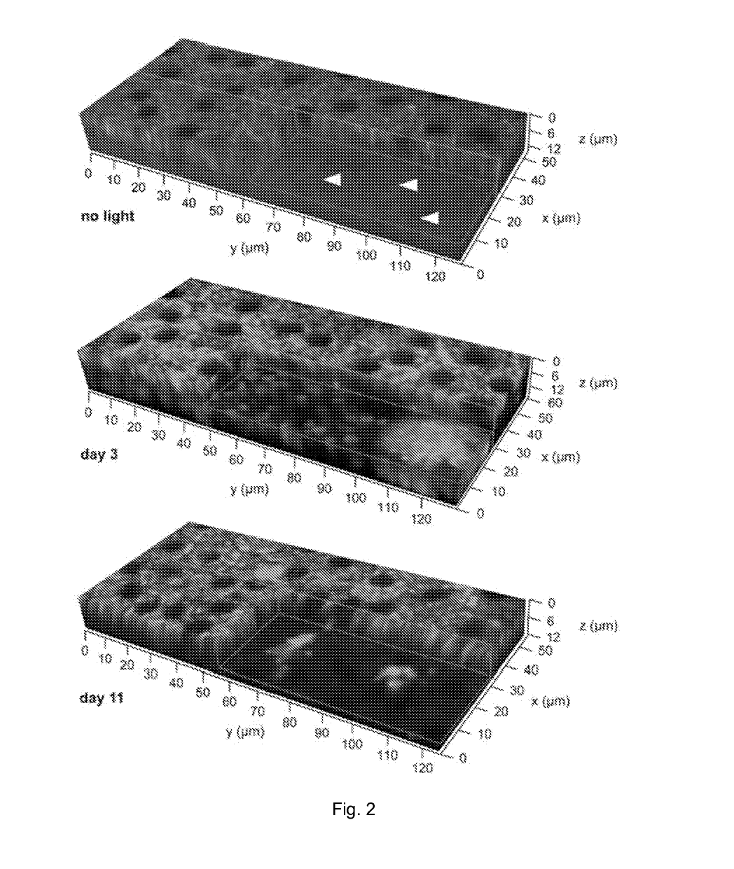

AF signals from 4-wk-old Abca4^{-/-}Rdh8^{-/-} mouse eyes (FIG. 2, Top) unexposed to light were uniformly distributed throughout the RPE cell layer as previously reported. However, here we also detected small uniformly distributed AF spots that appeared more like the tips of columns in our 3D reconstruction, extending from 8 μm under the RPE into the retinal space. Before light exposure, these AF spots were small and faint (FIG. 2, Top). At day 3 after light exposure (FIG. 2, Middle), irregular, larger, and brighter AF doughnut-like spots, most likely due to dying photoreceptors, were seen extending from 8 μm under the RPE into the retinal space. At about the same depth, we also detected AF granules with diameters over 25 μm, likely representing microglia/macrophages. At day 11 after light exposure (FIG. 2, Bottom), doughnut-like spots were no longer present, and the larger AF granules with varying round to elongated shapes were extended deeper in to the subretinal space.

Subretinal Translocation of Microglia in the Retinas of Abca4^{-/-}Rdh8^{-/-} Mice after Light Exposure

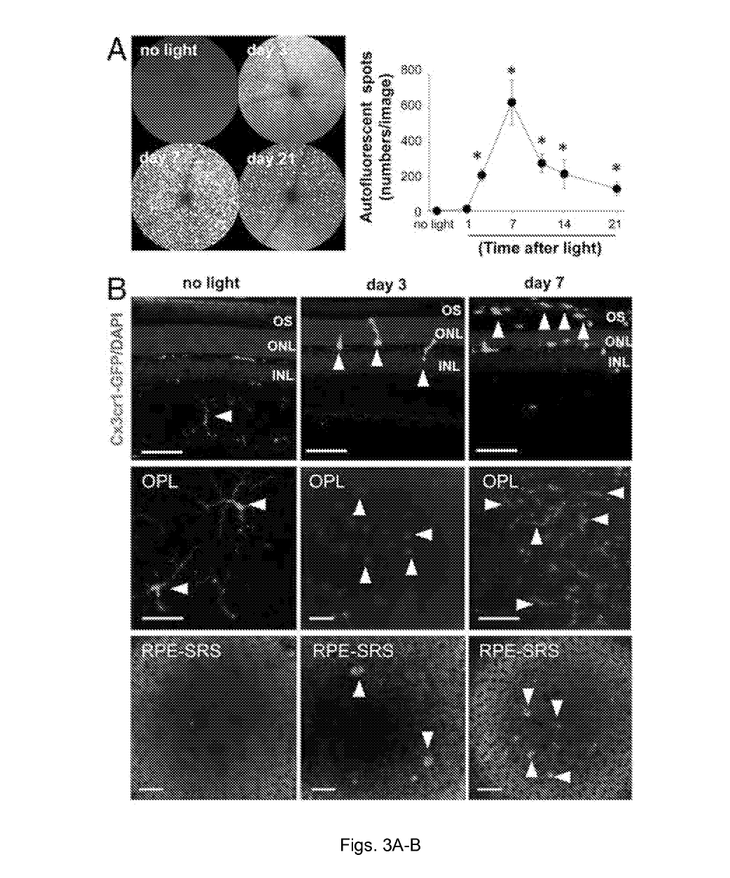

Damaged cells were largely cleared by day 11 (FIG. 1A). Thus, aided by a few initial clues, we explored possible mechanism(s) for this clearance. First was the supposition that the ˜25-μm diameter AF cells observed in the OS layer shown in FIG. 1A could represent infiltrating microglia/macrophages. Translocation of microglia/macrophages into the subretinal space is one of the features of retinal inflammation found in degenerating retinas. A second concern was that even though retinal infiltrating cells had been imaged by SLO as AF granules in Abca4^{-/-}Rdh8^{-/-} mice after light illumination, neither their fluorescence spectra nor their z location within the retina were known. Here we found increased numbers of SLO AF granules at day 3 that peaked at day 7 after light exposure [FIG. 3A, Left (images) and Right (quantification)]. To definitively identify the type of cell(s) infiltrating the RPE/retina junction, we then investigated microglial translocation in Cx3cr1Gfp/AAbca4^{-/-}Rdh8^{-/-} mice with microglia expressing GFP. Fluorescent imaging of their retinal sections after 10,000 l× light exposure for 60 min revealed microglia with round instead of ramified shapes translocated from the inner retina into the subretinal space (FIG. 3B, Top). These changes were also detected in intact mouse eyes by TPM imaging (FIG. 3B, Middle and Bottom). Before light exposure, only ramified GFP-expressing microglial cells were detected in the outer plexiform layer (FIG. 3B, Middle Left). At days 3 and 7, post-light-exposure changes observed by TPM in intact eyes included increased numbers of microglia with more-rounded shapes at the same locations (FIG. 3B, Middle Center and Middle Right). Round-shaped microglia cells also were more frequently detected in the subretinal space at days 3 and 7 after light exposure. Notably, infiltrating microglia in the subretinal space displayed a stronger AF intensity, probably due to their phagocytosis of OS debris (FIG. 3B, Bottom Center and Bottom Right).

### Light Exposure Alters Retinoid Metabolite Profiles of the Retina

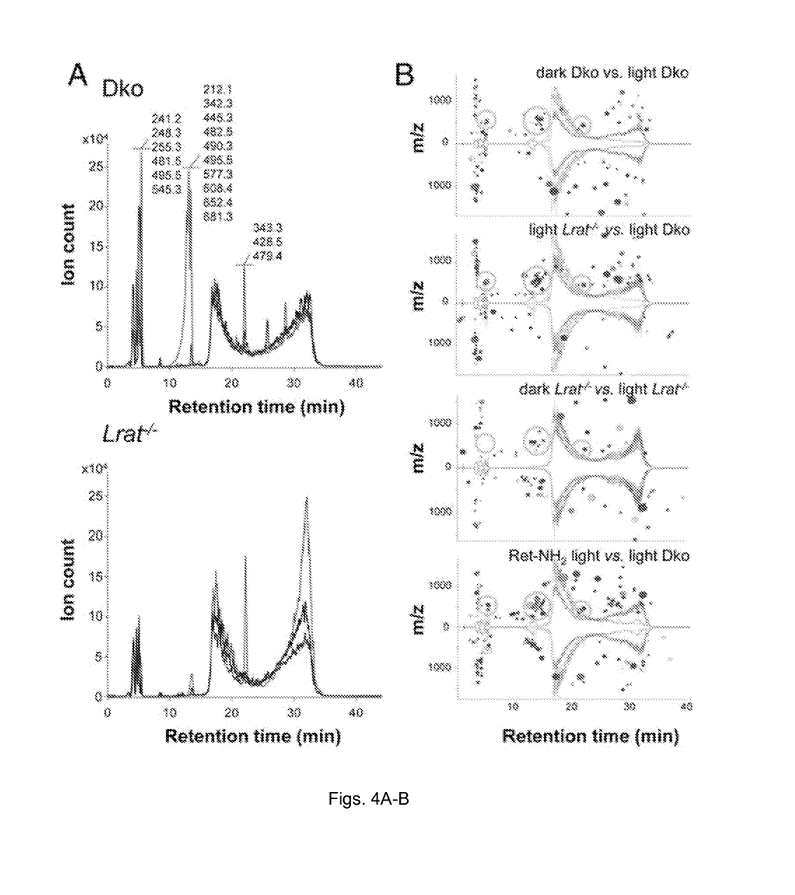

Fluorophores responsible for AF in the retina could be an indicator of global changes in the metabolic profile of this tissue. To evaluate and quantify these changes as well as determine whether they depend on a functional retinoid cycle, we used both genetically altered (Lrat^{-/-}) and pharmacologically treated (retinylamine) mice with metabolic profiles that were compared with light-exposed and dark-adapted Abca4^{-/-}Rdh8^{-/-} mice by using a LC/MS approach. Mouse retinas were isolated either on day 3 after light exposure (10,000 l× for 30 min) or from animals kept in the dark as controls. Metabolites were extracted with acetonitrile and subjected to MS analysis (FIG. 4). XCMS software was used to compare the data from individual samples that were grouped in analytically replicated datasets. Approximately 1,700 individual ions in the mass range of 200 to 2,000 m/z were aligned in these mouse retinal extract replicates. Almost 8% of all signals detected in these datasets demonstrated significant changes in their relative intensities (defined as a ≧1.5-fold change with P≦0.01). Notably, a given molecule could be represented by several different signals corresponding to differing isotopic distributions or nonspecific adducts. Nevertheless, these comparative data revealed that the most profound differences in ion composition between light-exposed and control retinal samples were detected during an HPLC retention period of 5-15 min and these included dramatic increases in several ion intensities in the mass range of m/z 220 to m/z 750 (FIG. 4A, Upper and FIG. 4B, Top). Additional ions with elevated intensities in light-exposed samples were observed at 22 min of elution. To investigate the origin of these light-induced alterations, we first analyzed the metabolic profile of retinal samples isolated from light-exposed lecithin:retinol acyltransferase (LRAT)-deficient mice that cannot produce visual chromophore. Here among 1,850 aligned individual ions, 174 showed significant signal changes. Although, there were spurious peaks most likely arising from differences in genetic background, the same set of ions eluted between 5 and 15 min that were identified previously in the light Abca4^{-/-}Rdh8^{-/-} (Dko) vs. dark Dko plot were clearly visible (FIG. 4A, Lower). However, importantly, unlike the peak with a retention time around 22 min, these ion signals did not appear in the differential plot of light-exposed vs. dark-adapted Lrat^{-/-} retinas (FIG. 4B), suggesting that they originated as a result of visual pigment activation. As an alternative to the above genetic approach, regeneration of visual chromophore in the eye was also markedly reduced by pretreatment of Abca4^{-/-}Rdh8^{-/-} mice with an inhibitor of the retinoid cycle, retinylamine, several hours before light exposure. Again, as noted with LRATdeficient samples, the most significant difference in the ion profiles occurred between 5 and 15 min of elution and involved an identical set of m/z values (FIG. 4B, Bottom). In summary, lightinduced alteration of the metabolic profile in the retina was dependent not only upon activation of functional visual pigment but also on its continuous effective regeneration with chromophore via the retinoid cycle. Thus, the observed effects can be linked to an excess of atRAL generated in photoreceptor cells. Although indirect, these data also support the idea that elevated atRAL levels play a critical role in retinal degeneration.

Changes in Photoreceptor OS at Day 1 after Light Exposure

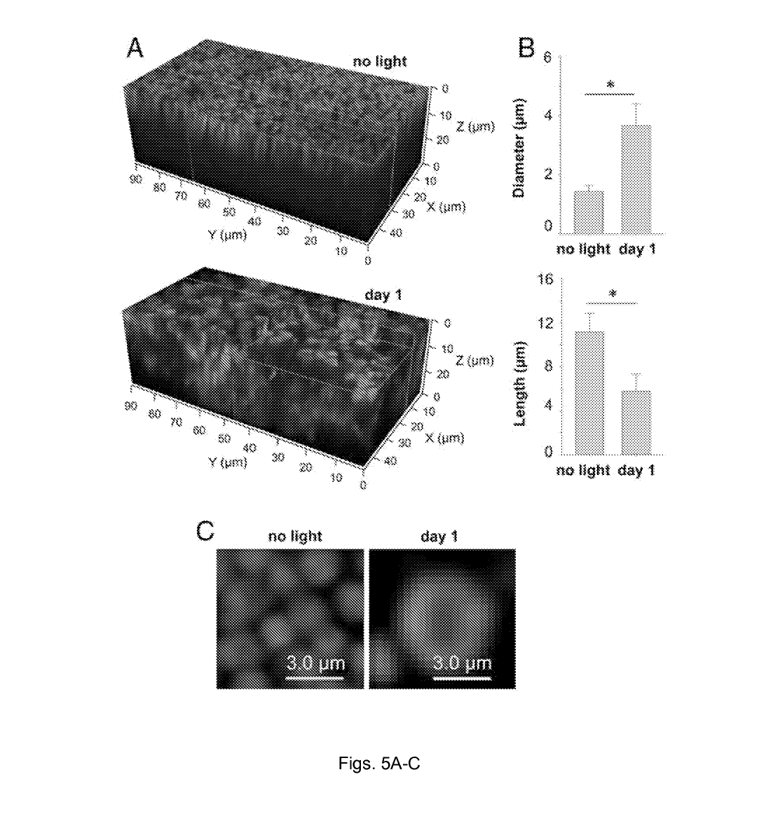

To obtain more detailed information about changes in the OS, we used TPM to image retinal tissues lacking the RPE ex vivo. Here, albino 4-wk-old Abca4^{-/-}Rdh8^{-/-} mice were exposed to light at 10,000 l× for 60 min and their retinas were harvested and stripped of the RPE at day 1 after light exposure. Such processed retinas were immediately analyzed by TPM. Photoreceptor OS in unexposed retinas lacking the RPE were uniformly distributed, showing a tight, regular arrangement (FIG. 5A, Upper). However, the OS of retinas at day 1 after light exposure displayed doughnutlike shapes, enlarged diameters, and shortened lengths (FIG. 5A, Lower and FIGS. 5 B and C). Measurements of these OS diameters were 3.67±0.73 μm in light-exposed mice and 1.43±0.19 μm in unexposed control animals.

Photoreceptor Cell Apoptosis is Caused by atRAL in Neural Retinal Tissue Culture

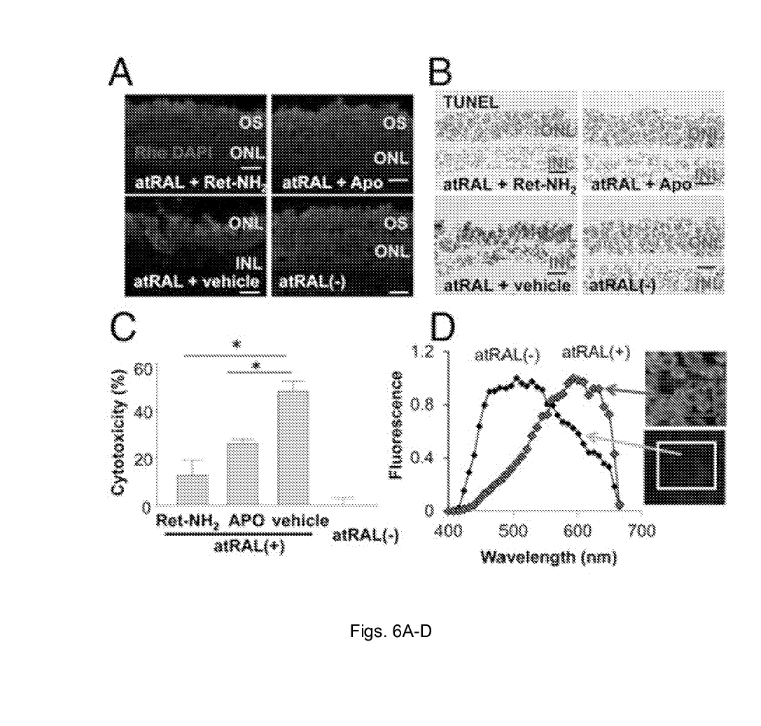

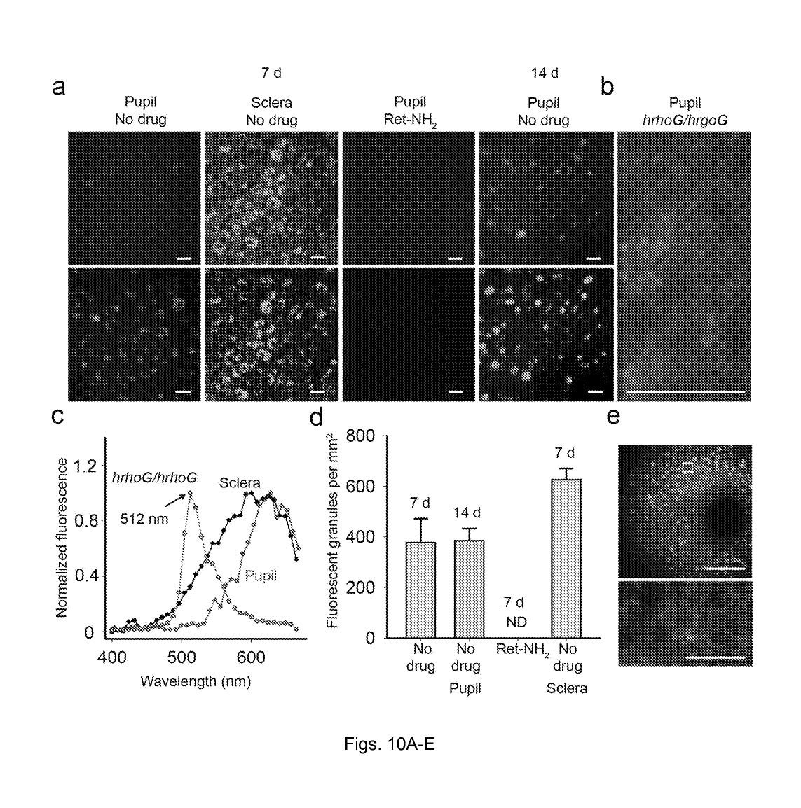

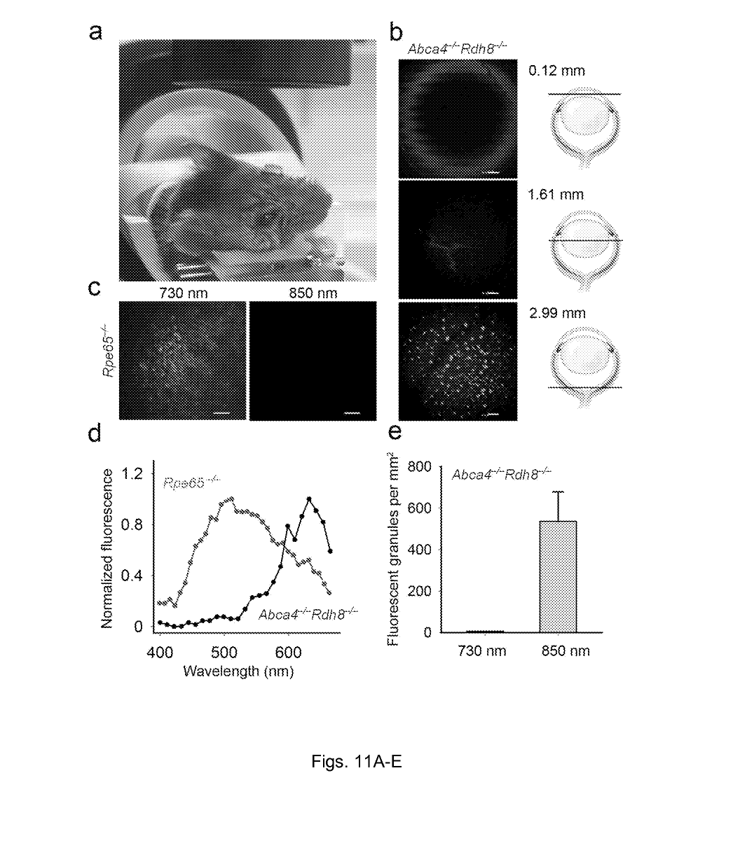

The primary cause of acute retinal degeneration after bright light exposure in Abca4^{-/-}Rdh8^{-/-} mice is the delayed clearance of atRAL from photoreceptors. Moreover, light-induced retinal degeneration in Abca4^{-/-}Rdh8^{-/-} mice can be prevented by pharmacological interventions such as the retinoid cycle inhibitor with a primary amino group, retinylamine, and the NAPDH oxidase inhibitor, apocynin (FIG. 10). Thus, to examine whether retinal tissue lacking RPE cells can display degenerative changes similar to those observed in in vivo, we performed ex vivo culture experiments with retinal explants coincubated with atRAL. Neural retinas were dissected from eyes of 4-wk-old WT mice, and incubated with 30 μM of atRAL in the presence of control vehicle, 30 μM of retinylamine or 300 μM of apocynin for 6 h at 37° C. Coincubation of atRAL with control vehicle resulted in marked retinal degeneration (FIG. 6A), and massive photoreceptor apoptosis was observed upon TUNEL staining (FIG. 6B). However, coincubation of atRAL with either retinylamine or apocynin prevented photoreceptor apoptosis in these retinal explant tissue cultures. Cell death rates were calculated by measuring lactate dehydrogenase released into tissue culture supernatants from dying cells. These calculated rates also indicated that atRAL-induced cell death in the neural retina was largely prevented by coincubation with retinylamine and apocynin (FIG. 6C), similar to results obtained in Abca4^{-/-}Rdh8^{-/-} mouse retina (FIG. 11).

Last, retinas of WT mice were incubated with 30 μM of atRAL for 24 h followed by TPM analysis. Notably, a spectrum similar to that of OS after light exposure in vivo (FIG. 1B) was obtained from the OS of retinal tissues incubated with atRAL (FIG. 6D, Left). Interestingly, the OS in retinal tissues incubated with or without atRAL showed enlarged diameters (FIG. 6D, Right), suggesting that retinal tissue culture conditions can induce OS damage as well. These results with the neural retinal tissue culture indicate that retinal degeneration is initiated by photoreceptor cell death independent of the RPE in Abca4^{-/-}Rdh8^{-/-} mice, and thus pathological changes in RPE cells appear to be secondary events. Furthermore, the same experiments also establish that TPM can reveal initial degenerative changes that occur in ROS.

RPE and ROS Changes in Abca4^{-/-}Rdh8^{-/-} Mice after Light Exposure

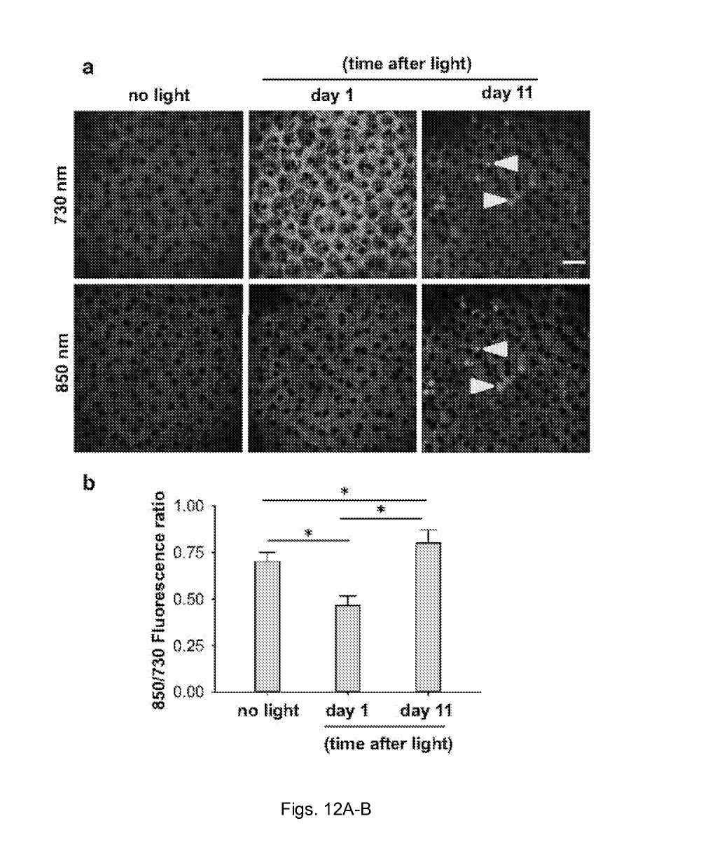

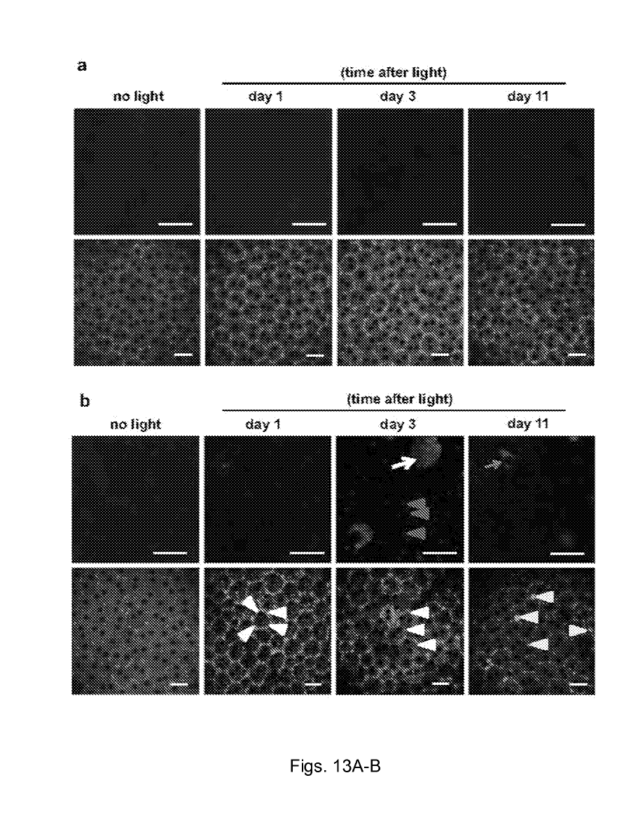

After bright light exposure, acute changes in OS over time were followed by changes in the RPE as shown by histological and immunocytochemical analyses. We studied the integrity of the RPE layer stained with an antibody against zonula occludentes (ZO-1), a resident protein of epithelial and endothelial cell membranes associated with tight junctions. Two weeks after bright light exposure (10,000 l× for 60 min), changes in RPE layer were observed in 6-wk-old Abca4^{-/-}Rdh8^{-/-} mice. Some RPE cells lost their expression of ZO-1 as indicated with the arrowheads in FIG. 12A. After light exposure, vertical cross-sections (FIGS. 12B and C) of the retina and horizontal sections (FIG. 12D) at the RPE level started to show changes, including a reduced size of cells and nuclei and a darker staining of cytosol, indicating cellular damage (FIG. 12E). Because RPE cells are postmitotic, they expand to fill space caused by RPE cellular defects. Shortened and disrupted OS and chromatin condensation in photoreceptor nuclei were observed at day 3 after light exposure (FIG. 13A). EM analyses revealed that photoreceptor cell debris included fragmented OS and IS between the RPE and outer nuclear layers of Abca4^{-/-}Rdh8^{-/-} mice at day 3 after light exposure (FIG. 13B). Fragmented photoreceptor debris and RPE cells also elicited AF signals from cryosections of retinas at day 3 after light exposure (FIG. 13C). Together these data suggest that light exposure results in a deterioration of RPE cells following changes in ROS.

AF Changes in Mertk^{-/-}Abca4^{-/-}Rdh8^{-/-} Mice

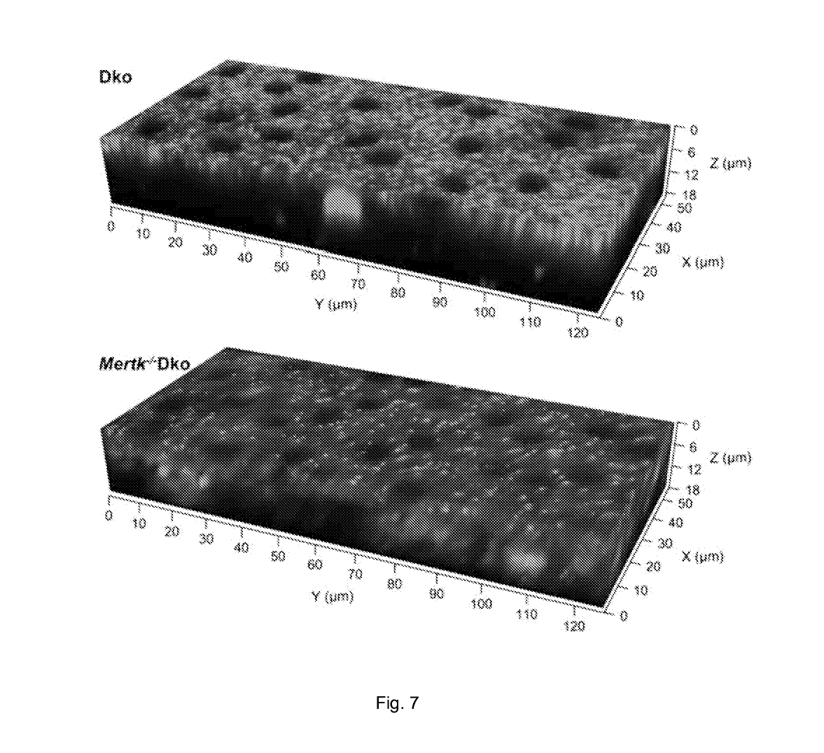

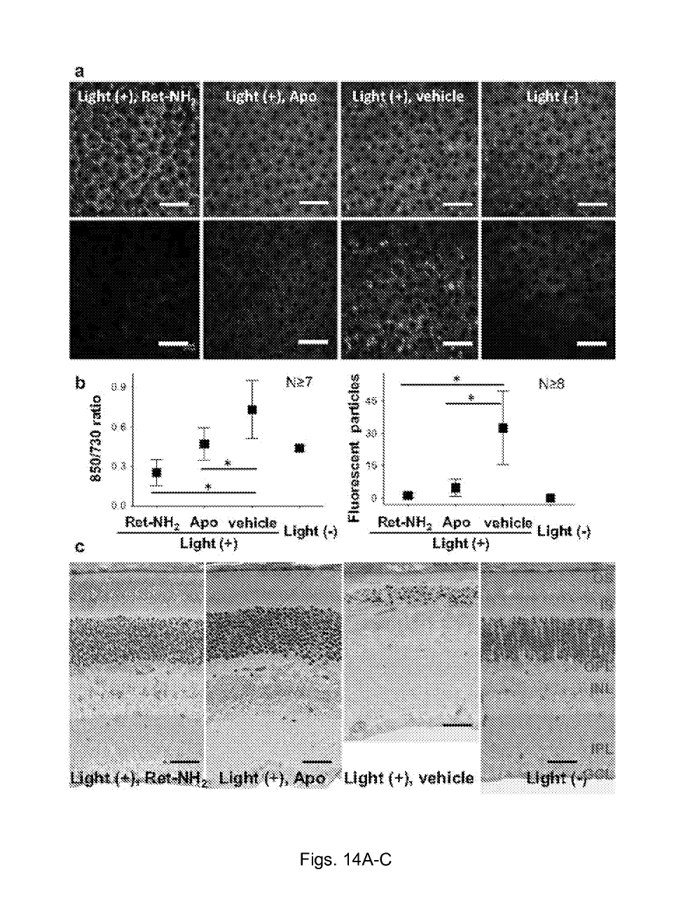

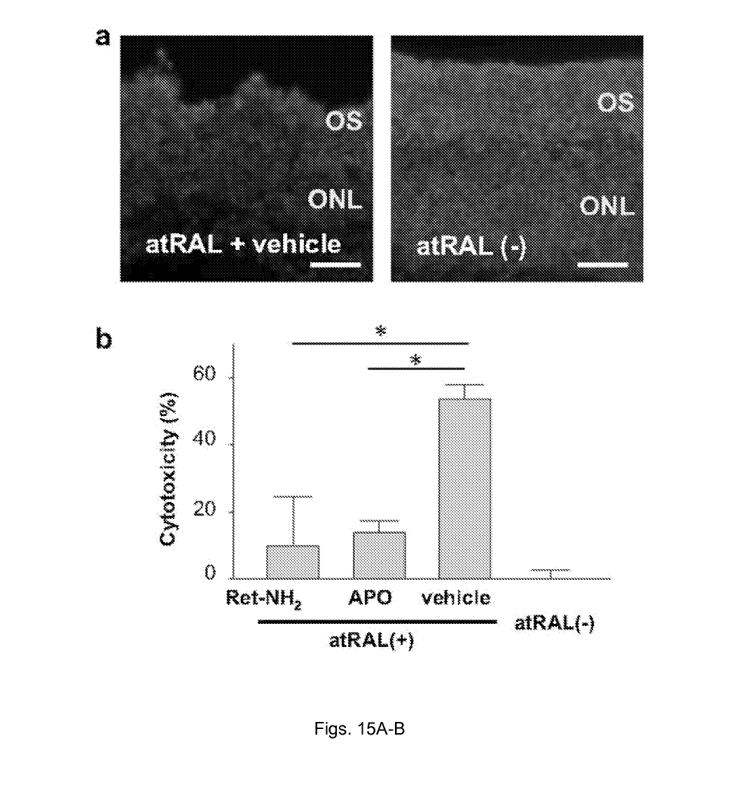

Finally, we used another genetic approach to probe light-induced degenerative changes in mouse retina. Phagocytosis of OS by the RPE is dramatically attenuated in Mertk^{-/-} mice. To determine whether retinal degeneration is initiated primarily by photoreceptor cell death in Abca4^{-/-}Rdh8^{-/-} mice, we investigated AF in retinas of Mertk^{-/-}Abca4^{-/-}Rdh8^{-/-} mice. Mertk^{-/-}Abca4^{-/-}Rdh8^{-/-} mice at the age of 3 wk were exposed to light at 10,000 l× for 60 min, and TPM analysis was performed at days 3 and 7 after exposure. TPM imaging of AF in the OS and RPE of intact eyes in Mertk^{-/-}Abca4^{-/-}Rdh8^{-/-} mice did not reveal any increase in the quantity of AF particles in the RPE compared with those seen in Mertk^{-/-}Abca4^{-/-}Rdh8^{-/-} mice that were not exposed to light (FIG. 7 and FIG. 14A). However, disrupted OS structures were noted even in mice unexposed to light (FIG. 14B), providing an early sign indicative of retinal degeneration. Retinal histology then was examined in Mertk^{-/-}Abca4^{-/-}Rdh8^{-/-} mice. Subretinal accumulation of photoreceptor debris was seen in plastic-embedded histological sections (FIG. 14C). Although light exposure caused photoreceptor damage and morphological sections manifested as decreased numbers of photoreceptors (FIG. 14C), accumulation of photoreceptor cell debris in the subretinal space was evident by both TPM and histological analyses even at day 7 after light exposure, despite the presence of microglia/macrophages (FIG. 15). Together, these observations indicate that AF particles that appear in the RPE at days 7 and 11 after light exposure in Abca4^{-/-}Rdh8^{-/-} mice could represent phagocytized materials from dead photoreceptor cells.

Here, we identified the sequence of changes in the retina that occurs as a consequence of exposure to brief strong illumination. Abca4^{-/-}Rdh8^{-/-} mice were used as an animal model that mimics fundamental changes in the retina relevant to human Stargardt disease and AMD. We provide clear evidence that the primary changes in the retina include retinoid-dependent formation of fluorescent metabolic by-products within rod photoreceptor cells, a nearly three-fold expansion/swelling of the ROS, and secondary infiltration of microglia/macrophages to clear photoreceptor cell debris. Finally, evidence is provided that phagocytosis-mediated transfer of retinoid adducts to the RPE is required to elicit damage to that cell layer.

Retinal inflammation is closely associated with the pathogeneses of human retinal diseases, including retinitis pigmentosa, Stargardt disease, and AMD. Moreover, infiltrating macrophages are thought to participate in the inflammation associated with retinal degeneration. Retinal macrophages are subdivided into tissue-resident microglia of the inner retina and peripheral macrophages that migrate to this site from retinal blood vessels. Recent studies suggest a pathogenic role for subretinal macrophages, even though they contribute to the clearance of photoreceptor cell debris. In this work, subretinal microglia/macrophages elicited an AF signal with a similar spectrum in both ROS and RPE cells, also suggesting subretinal microglia/macrophage involvement in clearing of ROS debris. This AF feature enabled the application of TPM and 3D reconstruction used in this study to monitor the sequence of events in the retinas of mice after bright light exposure (FIGS. 1, 2, 5, and 7 and FIGS. 12, 13, and 18).

We provide evidence that such products were formed in a retinoiddependent manner based on genetic considerations in conjunction with MS analyses (FIG. 4). Retinoids are highly reactive compounds prone to oxidation, isomerization, fragmentation, and condensation both with themselves and other membrane and protein components. atRAL along with its derivative products likely are the initiators of photoreceptor cell pathology for several reasons: (i) phototransduction is the only light-sensitive pathway in ROS and this process involves the conversion of retinoids and generation of atRAL; (ii) such light-induced degeneration can be prevented by pretreatment with retinoid cycle inhibitors (FIG. 14); and (iii) both retinoids and their condensation products are known to produce cellular toxicity and death. Retinal pathology could also result from mitochondrial dysfunction, because lipophilic unsaturated compounds such as retinoids can also act as electron acceptors that compromise ATP production.

For years it has been known that the lengths of ROS are reduced when animals are exposed to light for prolonged periods, but the molecular mechanism(s) remain obscure. Here, we observed that even though the length of ROS was reduced upon exposure to bright light, the volume of ROS expanded over approximately threefold. Advanced noninvasive TPM methods revealed swelling of ROS with increased AF disk diameters as early as 1 d after light exposure. Moreover, these changes in photoreceptor geometry were paralleled by differences in metabolic profiles of these retinas as determined by LC/MS at day 3 after exposure to light. Importantly, the imaging experiments were performed in a native setting with undisturbed intact eyes, which avoided potential artifacts arising from required tissue-processing. Although ROS sizes are known to be determined by rhodopsin content, these light-induced changes were too rapid for de novo protein biosynthesis to account for them. Additionally, it had been shown that rhodopsin mislocalized significantly to rod inner segments only at 48 h after light-induced damage. Thus, an osmotically driven influx of water after light exposure appears the most likely explanation for swelling of the ROS. Specifically without light exposure, the volume of the fluorescent portion of outer segments would be

πr²×h=3:14×0:715²×11:12=17:8 μm²;

whereas at day 1 after light exposure, the fluorescent portion of ROS was dramatically increased to

V=πr2×h=3:14×1:8352×5:73=60:6 μm2:

Thus, this expansion was not due to simple shortening of the compromised ROS. A plausible sequence of events could involve lower production of ATP, resulting in increased ion retention and osmotic pressure that in turn cause bursting of ROS followed by photoreceptor death. Previously it was reported that ATP insufficiency is correlated with the failure of the plasma membrane to maintain Ca²⁺ pump function with subsequent overaccumulation of Ca²⁺. Also it is known that photoreceptor cells die rapidly when retinas are incubated in medium deficient in glucose or other metabolites that fuel ATP biosynthesis.

We used ex vivo retinal tissue cultures to examine whether photoreceptor cells could degenerate without any contribution from RPE cells and found that coincubation of retinal tissues with atRAL caused photoreceptor cell apoptosis. Moreover, this apoptosis was prevented by coculture of retinal tissue with either retinylamine or apocynin, which also conferred protection against light-induced retinal degeneration in vivo. These experiments clearly identify photoreceptor cells as the primary targets for light-induced retinal degeneration and primary amine-mediated protection, but they do not exclude the RPE as a possible secondary target.

Detrimental actions of A2E accumulated in the RPE have been reported, including photosensitization and complement activation. However, it is not known whether these are a primary cause of retinal degenerative changes. Precursors of A2E formed in photoreceptor cell OS eventually reach RPE cells because the ends of the continuously renewed OS adjoining the RPE are removed by RPE cell phagocytosis. To examine the contribution of atRAL condensation products to retinal degeneration in Abca4^{-/-}Rdh8^{-/-} mice, we generated Mertk^{-/-}Abca4^{-/-}Rdh8^{-/-} mice that cannot carry out RPE phagocytosis. These mice still exhibited photoreceptor cell death without RPE phagocytosis after bright light exposure. Moreover, the RPE of these mice failed to display any AF changes, clearly indicating that A2E is not the primary initiator of light-induced retinal degeneration in this mouse model. However, Mertk-deficient mice did reveal infiltration of microglia/macrophages into the subretinal space, indicating that these cells likely contribute to the clearance of photoreceptor cell debris.

### Example 2

This Example describes two photon microscopy instances that can safely and periodically image the retina and RPE to detect and follow abnormalities in biochemical transformations well before electrophysiological and pathological changes become evident.

### Methods

### Mice

All animal procedures and experiments were approved by the Institutional Animal Care and Use Committee at Case Western Reserve University and conformed to recommendations of both the American Veterinary Medical Association Panel on Euthanasia and the Association for Research in Vision and Ophthalmology. B6(Cg)-Tyrc-2J/J mice were purchased from The Jackson Laboratory. Abca4^{-/-}Rdh8^{-/-} (DKO) and Rpe65^{-/-} mice were generated and genotyped as previously described. Human opsinGFP fusion, knockin hrhoG/hrhoG mice, expressing human rhodopsinGFP in photoreceptor outer segments were kindly provided by Dr. John H. Wilson (Baylor College of Medicine). All mice were housed in the animal facility at the School of Medicine, Case Western Reserve University, where they were provided with a regular mouse chow diet and maintained either under complete darkness or in a 12 h light (˜10 lux)/12 h dark cyclic environment. Euthanasia was performed in compliance with American Veterinary Medical Association (AVMA) Guidelines on Euthanasia, and approval by the Case Western Reserve University Institutional Animal Care and Use Committee. All mice used in this study were between 1-6-month-old. We used both male and female animals. DKO mouse pupils were dilated with 1% tropicamide prior to bright white light exposure at 10,000 lux (150 W spiral lamp, Commercial Electric) for 60 min After bright light exposure animals were housed in the dark until subsequent imaging sessions. Two-photon imaging to assess RPE and retinal changes was performed 7 and 14 days after bright light exposure.

Two-photon imaging was done through mouse eye pupil unless otherwise indicated, and either in vivo or with freshly enucleated mouse eyes for ex vivo imaging. For in vivo imaging, mice were anesthetized with an intraperitoneal injection of anesthetic solution consisting of ketamine (15 mg/ml), xylazine (3 mg/ml) and acepromazine 0.5 mg/ml diluted with water at a dose of 10 μl/g body weight (bw).

To enhance the visibility of retinosomes, WT mice without any drug treatment or treated with Ret-NH2 were exposed to 5,000 lux of white light for 5-30 min, 1 to 3 h before imaging.

### OCT

OCT imaging to verify retina integrity after TPM imaging was performed using SD-OCT Envisu R2200 (Bioptigen, Morrisville, N.C.)

### Retinylamine Treatment

Ret-NH2 was synthesized as described previously. Mice (4 to 6-week-old) were gavaged with 2 mg of Ret-NH2 solubilized in 100 μl soybean oil 13 to 16 h prior to bright light exposure. Two-photon imaging was performed 7 and 14 days after bright light exposure.

After treatment with Ret-NH₂ the content of fluorescent retinyl esters increases in the eye as reported previously. However, 7 days after treatment that increase has already diminished. For quantification of the impact of drug treatment, the same detector settings were used for mice that were treated and not treated with Ret-NH₂. This also applied to imaging with either 730 nm or 850 nm excitation. To prevent overload of the detector in this experiment, the settings were optimized to visualize condensation products (not retinyl esters), which were abundant in animals that were not treated with Ret-NH₂. This is why outlines of RPE cell borders are only very slightly visible in animals that were treated with Ret-NH₂.

The fluorescence intensity was brighter 14 days after light exposure than 7 days after exposure because it took some time for RPE cells to accumulate condensation products resulting from light exposure in mice that were not treated with Ret-NH₂.

### Two-Photon Imaging System for Mouse Retina and RPE

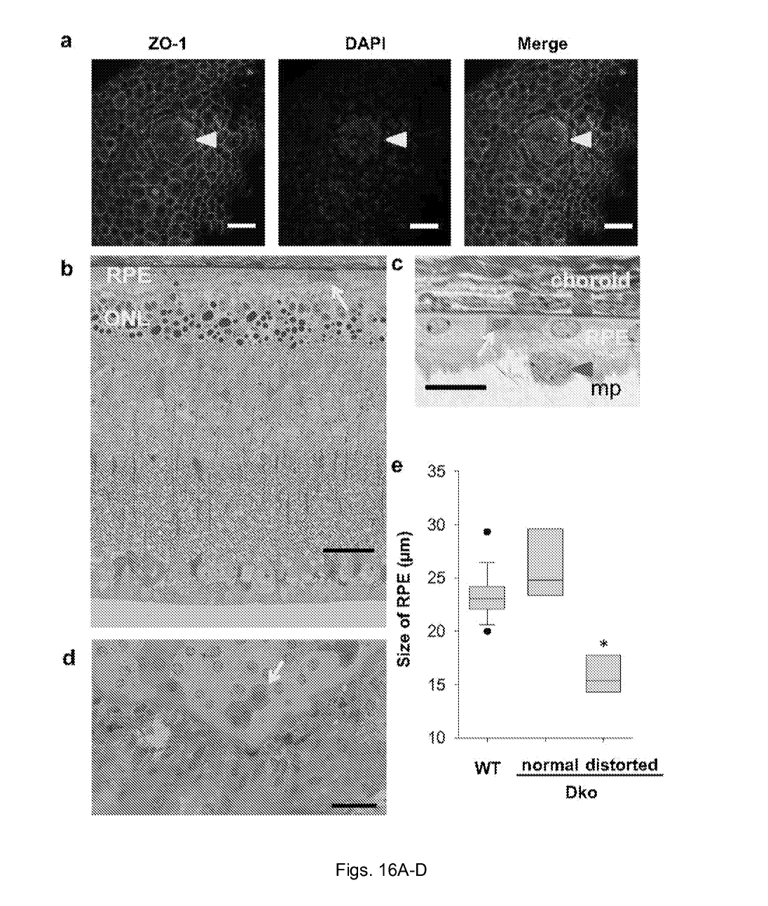

To achieve 2PE images of the retina and RPE with laser light entering through the mouse eye pupil, we modified the Leica (Wetzlar, Germany) TCS SP5 to include: an upright DM600 microscope stand, a Chameleon VisionS (Coherent, Santa Clara, Calif.) femtosecond laser, an objective with a 0.5 numerical aperture and 15 mm working distance, and a custom adaptive optics system including a deformable mirror (DM) (see FIG. 16a, 16b).

The tunable, 690-1050 nm, Chameleon VisionS generated 75 fs laser pulses at 80 MHz pulse repetition frequency. To minimize laser pulse duration at the sample, the laser was equipped with a group velocity dispersion precompensation (DC) unit with a 0 to 43,000 fs2 range. Laser beam power was controlled with an electrooptic modulator (EOM) contained within a safety box. After the EOM, the laser beam was directed to the adaptive optics component, namely DM, by the fold minor on a kinematic magnetic base (FMK1). The laser beam was coupled to the DM with expander lenses L1 and L2 (FIG. 16c). A microelectro-mechanical system DM (Boston Micromachines Corp., Cambridge Mass.) with 140 actuators, a 5.5 μm stroke, and gold coating provided fine focus adjustment and correction of aberrations introduced by the sample. In twophoton imaging, the excitation matters most because the emission fluorescence is generated only in the focal spot; therefore, it is critical to achieve a tightly focused excitation beam. Only the excitation light was modulated by the DM, which shape was controlled with software based on image quality metric feedback without the use of a wavefront sensor and associated components. This design reduced the cost of the system and its footprint. Lenses L3 and L4 reduced the size of the beam which, after reflecting off the second fold minor on a kinematic magnetic base (FMK2), was directed to the scan minors. The scan mirrors which operated with typical line frequency of 400 to 700 Hz and 512 to 1024 lines per frame, and typical pixel dwell time of 1.46 μs, were located at the plane conjugate to the back aperture of the 0.5 numerical aperture (NA) objective. In this configuration, the laser beam overfilled the mouse eye to take advantage of the NA of the dilated pupil. Laser power entering mouse pupil was 7.4 mW, based on an estimated 3.2 mm laser beam diameter and a 2 mm mouse eye pupil. We verified that estimate by placing a 2 mm iris at a location corresponding to the mouse eye pupil and measuring 8.5 mW using a laser power meter. Additionally, we measured that the needed laser light levels could be cut by over 25%. Only 6.3 mW of laser power was needed for imaging with this HYD detector, as compared to 8.5 mW of laser power required to obtain TPM images with the PMT detector. This represents over a 25% reduction in required laser power. This reduction is consistent with the HYD detector's higher quantum yield. At 500 nm, the quantum yield of the HYD detector was 45% as compared to the 27% quantum yield of the PMT R6357 detector used throughout the study. The fluorescence detector was located as close to the sample as possible to minimize loss of light available for image formation. Twophoton excited fluorescence leaving mouse eye pupil was collected by the same 0.5 NA lens, and directed to the photomultiplier tube (PMT) detector, Hamamatsu R6357, in a nondescanned manner after the excitation light was reflected off the dichroic minor (DCh) and filtered by the 680SPET Leica filter. 2PE spectra were obtained with a spectrally sensitive detector in a descanned configuration. For ex vivo imaging, the mouse eye was submerged in phosphatebuffered saline composed of 9.5 mM sodium phosphate, 137 mM NaCl and 2.7 mM KCl, and pH 7.4, with the pupil facing the excitation laser beam. For in vivo mouse imaging, the animal was surrounded by a heating pad and placed on a mechanical stage, which provided controlled movement around two rotational and in three translational axes (Bioptigen, Morrisville, N.C.). The mouse eye was covered with GenTeal gel that provided lubrication and refractive index matching with the RGP hard contact lens with a refractive index of 1.46, a radius of 1.7 mm and a flat front surface (Cantor and Nissel, Northamptonshire, UK). This contact lens directed laser light into the mouse eye, compensated for the refractive power of the corneaair interface, minimized the impact of corneal deformities and protected cornea from drying during the imaging session.

No changes to the cornea and lens were detectable using a low magnification sectioning microscope after completion of the imaging. Additionally, four weeks after TPM imaging of Rpe65^{-/-} mice, we used OCT to check for integrity of retinal layers. No differences were noted between mice that were imaged with TPM and control agematched Rpe65^{-/-} mice that were not imaged. Specifically, the outer nuclear layer average thickness in mice imaged with TPM was equal to 0.040 mm, with standard deviation of 0.002 mm, whereas corresponding measurements in control mice that were not imaged with TPM were 0.037 mm and 0.004 mm.

The scale bars displayed in the images were estimated by comparing measurements of en face TPM images of optic disks and histological sections.

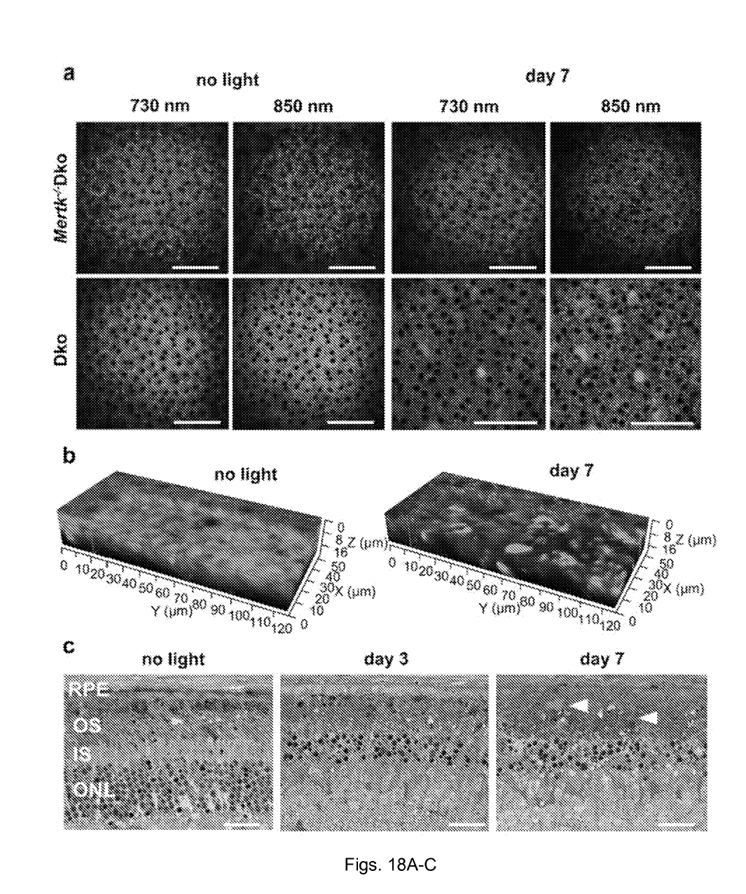

LAS AF Leica software and raw image data were used for quantification of fluorescent granules and fluorescence. Granules were counted in the inferior/central portion of the retina. The area selected was about 100 μm away from the edge of the optic disc. The RPE sampling area was kept between 0.05 mm2 to 0.1 mm2 for each eye. An example of the distribution of fluorescent granules around the optic disc is shown in FIG. 18e.

To calculate resolution along the optical axis (z-axis) as described in results referring to FIG. 9d, we used 730 nm excitation, the numerical aperture (NA) of the mouse eye equal to 0.4 and the coefficient of refraction of the vitreous humor equal to 1.33.

### Image Acquisition Algorithm

After focusing on the mouse RPE with a mechanical stage, optimization of the DM surface provided fine adjustments of focus and the excitation wavefront. Six Zernike modes were used as the set of basis functions for deformation of the DM surface. Zernike modes are a set of polynomials that are orthogonal to one another and frequently used to describe ophthalmic aberrations. The six modes used were Z0/2, Z2/2, Z2/2, Z1/3, Z1/3, Z0/4. The aberration compensation, (p, provided by the DM was Φ=ΣαjZj, where Zj is the Zemike mode with index j and the coefficient αj is the contribution of Zj. The coefficients were constrained such that −1.0<αj<1.0. The goal of optimizing the DM surface was to find a set of a coefficients which maximize the quality metric of a collected image. The quality metric used here was the normalized variance of the image.

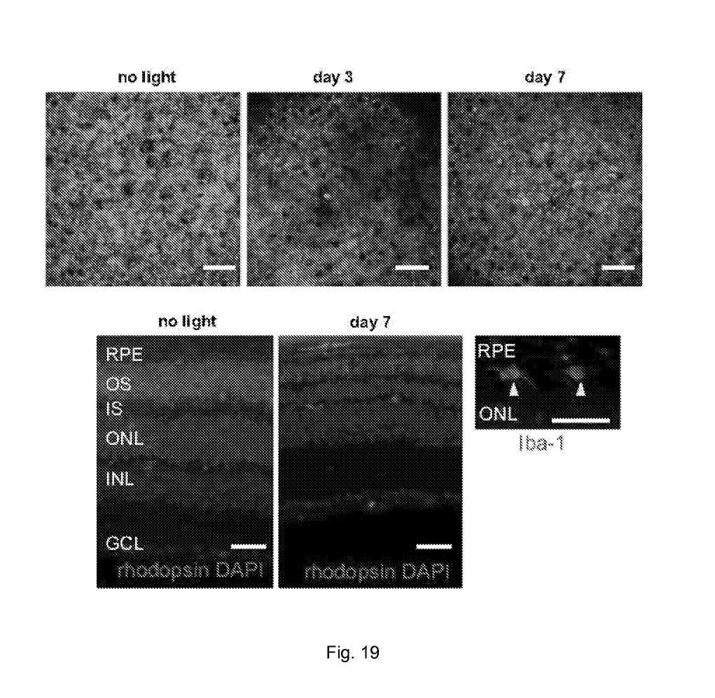

Optimization was performed by one of two procedures. In the first, the six Zernike modes were sequentially optimized Starting with focus, α₄, the coefficient, α₄ was varied from −0.9 to 0.72 in steps of 0.18 and the normalized variance was calculated at each step. The α₄ of the step which provided the best normalized variance value for the collected image was taken as the optimized coefficient for Z0/2. The was applied to the initially flat DM surface, and the procedure was repeated for the other aberration terms (Z2/2, Z−2/2, Z1/3, Z−1/3, Z0/4) such that the optimized Zernike modes accumulated on the DM surface. In the end, the vector of aj had been determined and the minor had accumulated the corresponding surface shape. This procedure was applied to image the hrhoG/hrhoG mice. The set of such established coefficients was: 0.72, −0.18, 0.00, 0.00, 0.18 and 0.00 for Zernike modes as listed above. The normalized variance of the image taken with these coefficients was 1717 versus the image collected with a flat mirror which had a normalized variance value of 246. This process collected 60 images and took 4-6 min to complete. However, the image with the best normalized variance value was not the image collected with the coefficients determined by the end of the process. The individual rod cells in hrhoG/hrhoG mice are difficult to distinguish initially without DM correction because of their small features. Sequential optimization was used to image hrhoG/hrhoG mice because each step is more independent of the previous one than in the second method described below. During sequential optimization, there is dependence on the previous steps because each subsequent Zernike mode builds off of the previous optimized Zemike mode. However, in the worst case, this would still provide at minimum 10 images with varying coefficients for defocus from which to choose. Here, the image with the best normalized variance (FIG. 10b) was collected during the defocus optimization stage, providing coefficients of −0.72, 0.00, 0.00, 0.00, 0.00, 0.00 and a normalized variance value of 1943. One possible reason why the image resulting from the complete sequential optimization was not the best in this case could be due to changes in the mouse eye itself, because this optimization was performed on a euthanized mouse. The second procedure for DM optimization is based on the stochastic parallel gradient descent (SPGD) method previously described. The normalized variance value, V, was calculated for an image collected using an initial set of a coefficients, specifically, all αj=0, which corresponds to a flat DM. Next, all αj were perturbed by a small amount, ζj, randomly chosen from between −0.05 and 0.05 in steps of 0.025, but excluding 0.0. This provided a new set of coefficients, αj+ζj. Using the new coefficients, a second image was collected, and the normalized variance was calculated to get Vζ. Starting coefficients for the next iteration (i) were then calculated as

$$α \frac{i + 1}{j} = {α}_{j} - n ζ j ( {V}_{ζ} - V ) ,$$

where η is the learning rate. Here a value of −0.01 was used for η, which is negative because the normalized variance was being maximized. The iterative process was performed for 40 steps and the DM surface that provided the largest normalized variance value was taken as optimal. This procedure was used to image live Rpe65^{-/-} mice (images shown in FIG. 19c). The optimization improved the normalized variance of 2374 for a flat DM to a value of 3024 for an optimized DM surface. The optimized coefficients for this mouse were −0.48, 0.05, 0.28, −0.08, −0.06, −0.24.

The sequential and SPGD optimization methods offer complementary approaches for improving image quality. The sequential optimization performs a search over a broad range of Zernike mode coefficients, which is useful if there are large aberrations or cells will be difficult to distinguish. If the features of interest are not resolved initially after sample preparation, the gradients needed by SPGD may be difficult to determine, but sequential optimization will systematically search and find coefficients that improve image quality. However, for sequential optimization, the search is coarse and the coefficients are not simultaneously optimized, in order to allow broad sampling within a reasonable time-frame. The SPGD performs gradient based optimization simultaneously for all Zernike modes. If the desired features can be resolved after initially localizing and focusing the sample, SPGD could more precisely determine the optimal coefficients compared to sequential optimization. However, SPGD requires the collection of more images, and therefore requires more time than sequential optimization. Therefore, based on the preparation and initial setup of the sample, one can decide whether SPGD or sequential optimization will be more appropriate, since eyes and aberrations differ greatly even within mice of the same genetic make-up.

### Statistical Analyses

Data in the bar graphs are expressed as the mean±S.D. The statistical analyses were carried out with ANOVA. Differences with P values >0.05 were considered not statistically significant.

### Results

### RPE Imaging Through a Mouse Eye Pupil

To image the RPE and retina in live mice we assembled an instrument containing a 75 fs laser with integrated group delay dispersion pre-compensation, adaptive optics modulating the excitation light and a fluorescence detector in a non-descanned configuration (FIG. 16a). Initial images of RPE created by endogenous fluorophores were obtained with ex vivo mouse eyes submerged in phosphate-buffered saline solution and a deformable mirror (DM) set to a neutral position (FIG. 16a, b, c). We optimized dispersion pre-compensation, which increased the mean fluorescence an average of 5-fold (FIG. 16d), indicating that in the RPE, 75 fs laser pulses would elongate to 400 fs. Iterative changes of the DM surface shape (FIG. 16e), resulted in further increased mean fluorescence from 34.6 to 58.1 in arbitrary units and increased dynamic range of the images, quantified as the range of pixel values, from 176 to 237 with 255 being the maximum (FIG. 160.

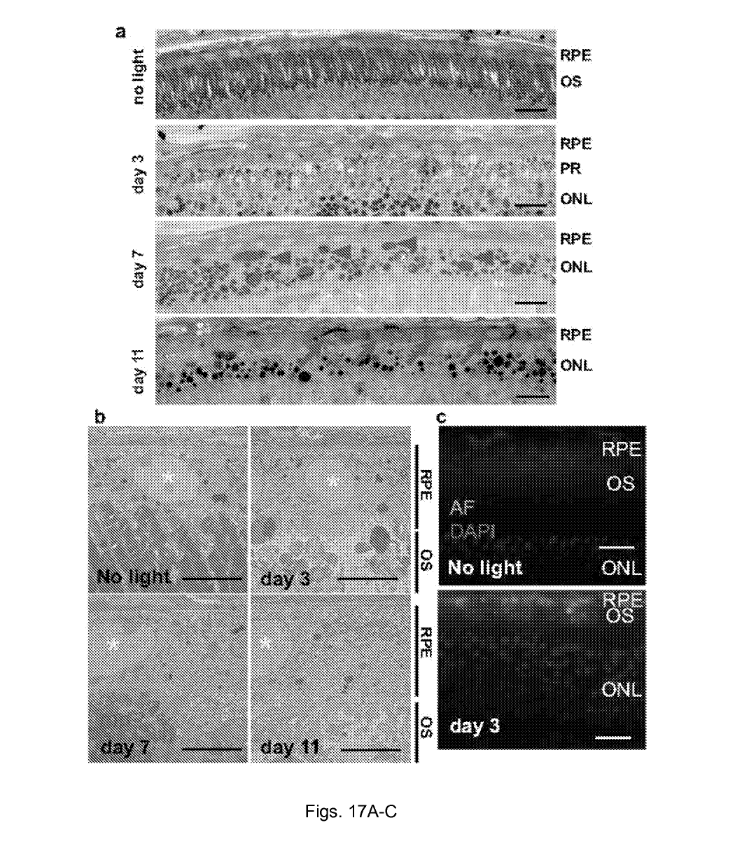

To assess the capabilities of our system to characterize the RPE and retina we imaged ex vivo eyes of mice with different genetic backgrounds. The brightest RPE images were obtained in Rpe65^{-/-} mice in response to 730 nm excitation (FIG. 17a). The brightly fluorescent granules correspond to enlarged retinosomes, which are a characteristic feature of the RPE only in Rpe65^{-/-} mice due to blockade of 11cisretinol synthesis. Double nuclei and retinosomes located close to individual cell membranes were also resolved (FIG. 17a). In contrast to Rpe65^{-/-} mice, predominant fluorophores in the RPE of Abca4^{-/-}Rdh8^{-/-} (DKO) mice were retinal condensation products. Fluorophores in these mice were more visible with an 850 nm excitation and were uniformly distributed within the RPE cell, so the black nuclei, free of fluorophores, were defined in TPM images (FIG. 17b). Retinosomes were visible in wild type (WT) mice exposed to white light for 30 min at 5,000 lux before imaging (FIG. 17c), and more clearly visible in WT mice pre-treated with retinylamine (Ret NH₂), a powerful inhibitor of the retinoid cycle 20, even though the laser power was reduced by 17% for the same detector settings. We also imaged and counted the neuronal nuclei in the ganglion cell layer at 0.6 mm eccentricity and found 2,500 nuclei per mm2 (FIG. 17d), which is smaller than previously reported about 7,000 per mm2 of combined ganglion and displaced amacrine cells in stained retina. This difference arises from: a) nuclei are free of fluorophores, and are only visible as dark structures against brighter cell bodies, which can lead to obstruction of the nuclei by axon bundles; b) not all the cell nuclei were at the same imaging depth; and c) the estimates of the area could be off by 40% because they were determined by comparing measurements of optic disk in en face TPM images to histological sections. Not all the cell nuclei were at the same location along the optical axis, the difference by only half of a ganglion cell soma diameter would place some of the somas out of TPM focus, because: a) the range of retinal ganglion cells somas diameters is 7-30 nm; b) the theoretical optical resolution along optical axis, estimated following Zipffel et al. was about 4.5 nm; and c) different layers of the retina come in and out of focus. Despite variances in absolute values of ganglion cell density, TPM-based visualization provides a non-invasive method for verification of the health of ganglion cell layer.

### Evaluation of Drug Therapy on RPE Preservation

Ret-NH2 protects mouse RPE and retina from deterioration caused by prolonged exposure to bright light. Using 2PE trans-pupil imaging ex vivo, 7 and 14 days after bright light exposure we found an over-accumulation of fluorescent granules in the RPE of untreated control DKO mice but no deposits in mice treated with Ret-NH₂ (FIG. 18a). These granules were more clearly visible when imaged with 850 nm rather than 730 nm light, indicating that they were condensation products of all-trans-retinal. Before we measured their emission spectra, we performed trans-pupil imaging of the retina of hrhoG/hrhoG mice (FIG. 18b) and determined that its emission maximum was at 512 nm. The spectra were almost identical with those obtained through the sclera and the previously published maximum at 511 nm Emission spectrum from granules in DKO mice had maximum at 628 nm. Even though slightly red-shifted, it is comparable with previous reports, confirming their origin as all-trans-retinal condensation products (FIG. 18c). Emission spectra obtained through the sclera showed a higher contribution of fluorophores emitting at shorter wavelengths, in agreement with brighter images obtained with 730 nm (FIG. 18a) as compared to trans-pupil, possibly caused by the spectral filtering introduced by the retina or anterior optics.

We counted the fluorescent granules; there were no differences in the quantity of fluorescent granules 7 days and 14 days after bleaching (FIG. 18d). Double nuclei and RPE cell borders are visible in the bottom panel of FIG. 18e.

### Localization of Bright Fluorescent Granules

Using a z-axis translation stage in our in vivo imaging system (FIG. 19a), we determined that the fluorescent granules responding to 850 nm excitation in live pigmented DKO mice exposed to bright light were located 3.0 mm away from the cornea (FIG. 19b). With 730 nm excitation we imaged retinosomes in live Rpe65^{-/-} mice 3.2 mm posterior to the cornea; differences likely result from mouse to mouse random variations. No fluorescence was observed in these mice using 850 nm light (FIG. 19c). The spectrum from the RPE of Rpe65^{-/-} mice was obtained with 730 nm and revealed maxima at 480 nm, 511 nm and a shoulder at 463 nm, whereas the spectrum from DKO mice was obtained with 850 nm and was shifted to longer wavelengths (FIG. 19d). The emission maxima at both 480 nm and 511 nm are likely generated by retinyl esters, whereas the shoulder at 463 nm is probably due to NADPH. The maximum around 511 nm could also be derived from all-trans-retinal, but the abundance of retinyl esters in Rpe65^{-/-} mice favors these retinoids as the primary source.

We counted on average 536 fluorescent granules per mm2 (FIG. 19e). The difference between ex vivo (FIG. 18d) and in vivo (FIG. 19e) was not statistically significant. The uneven edges of the cornea and lens sutures (FIG. 19b), corresponding to ˜145 breath/min of the mouse, result from using a slower acquisition rate for this image. Examination of TPM RPE images obtained during DM surface optimization did not indicate damage to RPE.

This example shows a) the first images of retinoid cycle fluorophores in RPE of living pigmented mammals and their spectral and spatial characterization; b) the first TPM images of rod photoreceptor cells; and c) the characterization of endogenous and artificial fluorophores in retina affected by genetic disorders, environmental stress or drug therapy.

TPM can be used to accelerate drug discovery and development by rapidly evaluating how compounds interact with tissues by determining their in vivo site(s) of action, as well as treatment safety and efficacy. Together with insights derived from parallel molecular, cellular and pathophysiological studies, TPM can foster effective treatment strategies for retinal diseases such as AMD, Stargardt disease and diabetic retinopathy. The cost effectiveness of using software driven adaptive optics will make TPM an attractive tool as therapeutic research transitions from mice to humans.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1-20. (canceled)

21. A method of determining the therapeutic effect of an agent on inhibiting retinal degeneration in a subject, the method comprising:
administering the agent to the subject;
irradiating the retina of the subject with short pulse light from a laser having a wavelength in the range of 600 nm to 1000 nm to stimulate two-photon induced fluorescence;
detecting two-photon induced fluorescence from inner and/or outer segments of the photoreceptor cells using a photon detector;
generating an image of the detected fluorescence in the inner and/or outer segments of the photoreceptors;
comparing the image to a reference image to assess the effect of the agent on inhibiting photoreceptor cell death.

22. The method of claim 21, wherein a decrease in the amount or spatial localization of the fluorescence of the generated image compared to the reference image is indicative of the compound inhibiting photoreceptor cell death.

23. The method of claim 21, further comprising generating a three dimensional image of the photoreceptor outer segment based on the detected fluorescence to determine the shape and/or volume of the outer segment of the photoreceptor and to assess the effect of the agent on inhibiting photoreceptor cell death.

24. The method of claim 23, wherein a decrease in volume of the photoreceptor outer segment compared to a reference volume is indicative of the agent inhibiting photoreceptor cell death.

25. The method of claim 21, wherein the light used to irradiate the retina has a wavelength in the range of about 710 nm to about 750 nm.

26. The method of claim 21, wherein the subject is a human.

27. The method of claim 21, wherein the subject is a genetically engineered animal.

28. The method of claim 21, wherein the subject is an Abca^{-/-}Rdh8^{-/-} mouse.

29. The method of claim 21, wherein the retina of the subject is irradiated with light effective to induce retinal degeneration prior to irradiating the retina to stimulate two photon induced fluorescence.

30. The method of claim 29, wherein the retina of the subject is photobleached prior to irradiating the retina to stimulate two photon induced fluorescence.

31. The method of claim 21, wherein laser is directed to a deformable mirror prior to irradiating a focal volume of the retina, wherein the deformable mirror provides fine focus adjustment and aberration correction of the laser on focal volume of the retina.

32. The method of claim 31, wherein the shape of the deformable mirror is controlled by an image quality metric feedback without the use of a wavefront sensor.

33. The method of claim 12, wherein a plurality of Zernike nodes are used as basis functions for deformation of the deformable mirror and focus and excitation of the laser.

34. The method of claim 33, wherein the Zernike nodes are sequentially optimized.

35. The method of claim 33, wherein the Zernike nodes are optimized using a stochastic parallel gradient descent method.

36. The method of claim 31, wherein irradiating the retina of the subject with light from the laser comprises irradiating the retina with light having a pulse length in the range of 10 fs to 100 fs.

37. The method of claim 31, wherein irradiating the retina of the subject with light from the laser comprises irradiating the retina with a laser with a repetition frequency in the range of 76 Mhz to 100 MHz.

38. The method of claim 1, wherein the agent comprises at least one of a Gs or Gq coupled serotonin receptor antagonist, an alpha 1 adrenergic antagonist, an alpha-2 adrenergic receptor agonist, and adenylyl cyclase inhibitor, an M3 receptor antagonist, a PLC inhibitor, or a primary amine, which forms transient shiff-bases with all-trans retinal in the eye.

39-57. (canceled)

58. A method of determining the therapeutic effect of an agent on inhibiting retinal degeneration in a subject, the method comprising:
administering the agent to the subject;
irradiating the retina of the subject with short pulse light from a laser having a wavelength in the range of 600 nm to 1000 nm to stimulate two-photon induced fluorescence of retinoid cycle fluorophores of the retinal pigment epithelium (RPE);
detecting two-photon induced fluorescence of retinoid cycle fluorophores of the retinal pigment epithelium (RPE) using a photon detector;
generating an image of the detected fluorescence of the retinoid cycle fluorophores of retinal pigment epithelium (RPE);
comparing the image to a reference image to assess the effect of the agent on inhibiting retinal degeneration.

59. The method of claim 58, wherein an increase in the amount or spatial localization of the fluorescence of the generated image compared to the reference image is indicative of an increased risk of retinal degeneration.

60-87. (canceled)
